SEQUENCE LISTING (20 sequences)

SEQ ID NO: 1 - DNA, 101 bp, Homo sapiens
ccctcccactggctgggttccagcagtcggtggcaggtgggcaggcgcctgcttctgggcggggatcatgtcgtcaatggactggcctttctccaacttct

SEQ ID NO: 2 - DNA, 101 bp, Homo sapiens
ccctcccactggctgggttccagcagtcggtggcaggtgggcaggcgcctacttctgggcggggatcatgtcgtcaatggactggcctttctccaacttct

SEQ ID NO: 3 - DNA, 101 bp, Homo sapiens
agaagttggagaaaggccagtccattgacgacatgatccccgcccagaagcaggcgcctgcccacctgccaccgactgctggaacccagccagtgggaggg

SEQ ID NO: 4 - DNA, 101 bp, Homo sapiens
agaagttggagaaaggccagtccattgacgacatgatccccgcccagaagtaggcgcctgcccacctgccaccgactgctggaacccagccagtgggaggg

SEQ ID NO: 5 - DNA, 101 bp, Homo sapiens
agcccccgtggcgatccgagatgatggggtcaaagagttccttgaaaacttcgtaggactcctcatcaccagccacgcagcccacggtcatgatgaagggg

SEQ ID NO: 6 - DNA, 101 bp, Homo sapiens
agcccccgtggcgatccgagatgatggggtcaaagagttccttgaaaactccgtaggactcctcatcaccagccacgcagcccacggtcatgatgaagggg

SEQ ID NO: 7 - DNA, 101 bp, Homo sapiens
ccccttcatcatgaccgtgggctgcgtggctggtgatgaggagtcctacgaagttttcaaggaactctttgaccccatcatctcggatcgccacgggggct

SEQ ID NO: 8 - DNA, 101 bp, Homo sapiens
ccccttcatcatgaccgtgggctgcgtggctggtgatgaggagtcctacggagttttcaaggaactctttgaccccatcatctcggatcgccacgggggct

SEQ ID NO: 9 - DNA, 101 bp, Homo sapiens
cgaggtcatgttccccctccttgtacagaacgaatatgtcatagccgacaccagagcgacactgcagggtcaggctgcctccgcgggccacgacagagccc

SEQ ID NO: 10 - DNA, 101 bp, Homo sapiens
cgaggtcatgttccccctccttgtacagaacgaatatgtcatagccgacatcagagcgacactgcagggtcaggctgcctccgcgggccacgacagagccc

SEQ ID NO: 11 - DNA, 101 bp, Homo sapiens
gggctctgtcgtggcccgcggaggcagcctgaccctgcagtgtcgctctggtgtcggctatgacatattcgttctgtacaaggagggggaacatgacctcg

SEQ ID NO: 12 - DNA, 101 bp, Homo sapiens
gggctctgtcgtggcccgcggaggcagcctgaccctgcagtgtcgctctgatgtcggctatgacatattcgttctgtacaaggagggggaacatgacctcg

SEQ ID NO: 13 - DNA, 101 bp, Homo sapiens
ctttggttggtgccctgatcccaccctcggtgggcccacaggttcccccattccctgctcacccaatgtcctgtgtttgctctgacgccgacattggagga

SEQ ID NO: 14 - DNA, 101 bp, Homo sapiens
ctttggttggtgccctgatcccaccctcggtgggcccacaggttcccccagtccctgctcacccaatgtcctgtgtttgctctgacgccgacattggagga

SEQ ID NO: 15 - DNA, 101 bp, Homo sapiens
tcctccaatgtcggcgtcagagcaaacacaggacattgggtgagcagggaatgggggaacctgtgggcccaccgagggtgggatcagggcaccaaccaaag

SEQ ID NO: 16 - DNA, 101 bp, Homo sapiens
tcctccaatgtcggcgtcagagcaaacacaggacattgggtgagcagggactgggggaacctgtgggcccaccgagggtgggatcagggcaccaaccaaag

SEQ ID NO: 17 - DNA, 101 bp, Homo sapiens
aggaagagaaaacgatgtctagcaatagcccaagaggtgagtagctgaacgttttatagagatgaggagagactaactaaggactagggcgcatcccttta

SEQ ID NO: 18 - DNA, 101 bp, Homo sapiens
aggaagagaaaacgatgtctagcaatagcccaagaggtgagtagctgaacattttatagagatgaggagagactaactaaggactagggcgcatcccttta

SEQ ID NO: 19 - DNA, 101 bp, Homo sapiens
taaagggatgcgccctagtccttagttagtctctcctcatctctataaaacgttcagctactcacctcttgggctattgctagacatcgttttctcttcct

SEQ ID NO: 20 - DNA, 101 bp, Homo sapiens
taaagggatgcgccctagtccttagttagtctctcctcatctctataaaatgttcagctactcacctcttgggctattgctagacatcgttttctcttcct

(19) United States
(12) Patent Application Publication
(10) Pub. No.: US 2016/0230231 A1
(43) Pub. Date: Aug. 11, 2016

(54) GENOTYPING TESTS AND METHODS FOR EVALUATING PLASMA CREATINE KINASE LEVELS

(71) Applicant: INSTITUT DE CARDIOLOGIE DE MONTREAL, Montreal (CA)

(72) Inventors: Marie-Pierre Dube, Montreal (CA); Jean-Claude Tardif, Laval (CA)

(21) Appl. No.: 15/029,387

(22) PCT Filed: Oct. 17, 2014
(86) PCT No.: PCT/IB2014/065427

§ 371 Date: Apr. 14, 2016

Related U.S. Application Data
(60) Provisional application No. 61/892,895, filed on Oct. 18, 2013.

(51) Int. Cl. C12Q 1/68

(52) U.S. Cl. CPC C12Q 1/6883; C12Q 2600/106; C12Q 2600/156; C12Q 2600/16

(57) ABSTRACT

The invention relates to genetic variants useful for evaluating creatine kinase levels in a subject and determining an Nupper limit of normal (ULN) CK level for a subject. ULN CK level is used in determining the pathological significance of measures of blood or plasma CK obtained from the subject. The methods and compositions of the invention are useful for providing a genetic-C ally determined or individualized ULN CK level, for diagnosing statin-induced myopathy and for providing statin therapy.

### FIELD OF THE INVENTION

The present invention relates to the general field of pharmacogenomics and is particularly concerned with the use of specific genetic variants in the evaluation of non-pathological or pathological plasma CK levels in a patient and in diagnosis of statin-induced myopathy.

### BACKGROUND OF THE INVENTION

Statins (HMG-CoA reductase inhibitors) are the most prescribed class of lipid-lowering drugs used in the treatment and prevention of cardiovascular disease. Despite reducing clinical cardiovascular events by 20 to 50% (Vaughan C J, Gotto A M, Circulation. 2004; 110:886-892) and having a positive benefit to risk ratio, statins are underutilized (Kotseva K et al, Eur J Cardiovasc Prey Rehabil. 2009, 16:121-137 and Cardinal H et al, Pharmacoepidemiol Drug Saf. 2006, 15:57-61) as they can cause muscular side effects ranging from non-specific myalgia to rhabdomyolysis (Harper et al Curr Atheroscler Rep. 2010, 12:322-330). The frequency of muscle symptoms was evaluated at 10.5% in the Prediction of Muscular Risk in Observational Conditions (PRIMO) study, an observational study in an unselected hyperlipidemic patient population receiving high-dose statins (Brukert E, et al Cardiovasc Drugs Ther. 2005, 19:403-414). Many patients stop statin therapy for aches and pains that are mistakenly believed to be related to their medication, underscoring the need for improved clinical tools for diagnosing statin-induced myotoxicity. Clinicians often measure serum creatine kinase (CK) as a proxy for the severity of statin-induced myotoxicity, but interpretation of the pathological significance of CK level, in diagnosis of statin-induced myopathy is not straight forward (Brancaccio P, et a/Br Med Bull. 2007, 81-82:209-23; Pasternak R C, et al Circulation. 2002, 106:1024-1028; Sewright K A, et a/Curr Atheroscler Rep. 2007, 9:389-396).

Creatine kinase (CK) catalyzes the reversible transfer of high energy phosphates between ATP/ADP and creatine systems. CK is important for normal energy homeostasis and exerts several integrated functions, including temporary energy buffering, metabolic capacity, energy transfer and metabolic control. CK level and activity are clinically important and CK serves as a biomarker for several diseases including statin-induced myopathy, rhabdomyolysis, myocardial infarction, muscular dystrophy and autoimmune myositis.

Serum CK levels exceeding three times the upper limit of normal (ULN) often lead to changes in statin therapy (change in dose or drug, or withdrawal of treatment altogether). However, the normal range of serum CK concentrations observed in individual patients and between patients is wide, limiting the utility of serum CK as a diagnostic biomarker. Variables such as gender, ethnicity and age are highly correlated with serum or blood CK levels. Women tend to have lower baseline CK levels and may respond differently to physical activity (Amelink G J, et al 1990 Acta physiologica Scandinavica. 138:115-124; Komulainen J, et al 1999 Acta physiologica Scandinavica 165:57-63; Rinard J, et al 2000 J Sports Sci. 18:229-236; Clarkson P M, et al 2002 Am J Phys Med Rehabil. 81:S52-69).

Statins can cause a wide range of muscular side effects with no specific clinical characteristics, from non-specific myalgias to rhabdomyolysis, with symptoms usually developing within four weeks but can be delayed up to four years after statin initiation. Noncompliance is thought to be due to aches and pains that are mistakenly identified by patients and physicians as statin-induced myopathy. In fact clinical data indicates that statin-induced myopathy occurs in only 7-10% of patients and has life-threatening complications in only 0.001% (Bruckert E., et al Cardiovasc Drugs Ther. 2005, 19:403-414). Thus, 40% of patients who stop taking statin do so for the wrong reason and would have benefited had they continued to take the drug. Statins are unfortunately underused due to misdiagnosis of statin-induced myopathy.

Diagnosis of statin induced myopathy is typically based on the presence of muscle related symptoms and measures of serum levels of creatine kinase (CK). CK is an enzyme marker of muscle breakdown that is used as a surrogate to detect muscle damage. Typically elevated levels of CK are considered diagnostic of statin-induced myopathy including myositis, myalgia and rhadomyolysis. Clinicians typically use serum creatine kinase (CK) levels, as a rough proxy for severity of statin-induced myotoxicity, but the correlation between symptoms and CK level is not well established.

Normal or non-pathological levels of CK are highly variable between individuals. Currently what is considered a ‘normal’ CK level is generally between 10 and 150 U/L with men typically having higher levels than females. Furthermore, some individuals with elevated CK i.e. 3 to 10-fold above the ULN CK threshold (150 U/L) do not experience statin-induced myopathy and others without elevated CK i.e. less than 3-fold below the ULN do experience statin-induced myopathy (Goldenberg N and Glueck C J., Vasc Health Risk Manag. 2009). As a result elevated CK level and muscle symptoms are often not sufficient for diagnosis of statin-induced myopathy and muscle biopsy is often needed to provide evidence of myotoxicity.

Elevations less than threefold above CK upper limit of normal (ULN) are typically considered of little consequence i.e. <450 U/L. Conversely, clinicians often intervene in statin therapy (change dose or change drug) when an individual's serum CK levels exceeds threefold the ULN i.e. >540 U/L. At present, best available practice supports three diagnostic strata: (i) incipient myopathy (CK 3-fold above the ULN and less than 10-fold above the ULN), (ii) myopathy (CK 10-fold above the ULN and less than 50-fold above the ULN), and (iii) rhabdomyolysis (CK above 50-fold the ULN). CK levels are not routinely measured before statin therapy begins. When CK levels are elevated above the ULN threshold, the statin is usually withdrawn, although it is difficult to determine whether statin therapy or another cause is to blame.

As a result of these complexities there is yet no consensus on the definition of statin myopathy and related conditions. The American College of Cardiology (ACC), American Heart Association (AHA), National Heart, Lung and Blood Institute (NHLBI) (Pasternak, R. C. et al. 2002) the FDA (Sewright, K. A. et al. Curr Atheroscler Rep. 2007, 9:389-396) and National Lipid Association (NLA) (McKenney, J. M., et al, Am J Cardiol. 2006, 97:89C-94C) have each proposed different definitions for statin-related muscle effects as illustrated in Table 1. This lack of consensus around the definition of statin myopathy contributes to misdiagnosis and hinders estimation of the true incidence of statin-induced myopathy.

TABLE 1
Definitions of Statin-Induced Myopathy

| Condition | ACC/AHA/NHLBI 2002 | NLA 2006 | FDA |
|---|---|---|---|
| Myopathy | General term | Complaints of | CK ≧ 10 × |
| | referring to | myalgia (muscle | ULN |
| | any disease | pain or soreness), | |
| | of muscles | weakness, and/or | |
| | | cramps, plus | |
| | | elevation in serum | |
| | | CK > 10 × upper | |
| | | limit of normal | |
| | | (ULN) | |
| Myalgia | Muscle ache or | NA | NA |
| | weakness without | | |
| | CK elevation | | |
| Myositis | Muscle symptoms | NA | NA |
| | with increased CK | | |
| Rhabdo- | Muscle symptoms | CK > 10,000 IU/l | CK > 50 × |
| myolysis | associated with | or CK > 10 × ULN | ULN and |
| | marked CK | plus an elevation | evidence of |
| | elevations, typically | in serum creatine | organ dam- |
| | substantially >10 × | or medical | age, such |
| | ULN and with | intervention with | as renal |
| | creatine elevation | i.v. hydration | compromise |

Pharmacogenomics could provide tools for better diagnosis of statin-induced myopathy and some relevant pharmacogenomic associations are known. Variation in the SLCO1B1 gene is known to be associated with risk of developing statin-induced myopathy, in particularly with administration of simvastatin (Peters B, et al, 2009 Genome Med 1, 120.). The pharmacokinetics of fluvastain is known to be influenced by CYP2C9 genotype (Kirchheiner J, et al, 2003 doi:10.1038/clpt.2010.274). CYP3A5 genotype has been associated with CK levels and muscle damage in patients taking either atorvastatin or simvastatin (Wilke R, et al, 2005 Pharmacogenetics and Genomics 15, 415-42). A number of genetic factors have also been associated with increases statin muscle concentration in patients taking statins including variants of the genes: CYP2D6, CYP3A4, CYP3A5, GATM, SLCO1B1, ABCB1 and ABCG2 (Canestaro W J, et al 2014 Genetics in Medicine. doi:10.1038/gim.2014.41)

As such there is a need for more reliable and accurate diagnosis of statin-induced myopathy which could help to ensure compliance in a larger percentage of the population treated, reduce wasteful spending and increase the overall clinical benefit derived. An object of the present invention is therefore to provide methods, reagents and kits for improved diagnosis of statin induced myopathy.

### SUMMARY OF THE INVENTION

The present invention relates to methods, compositions, reagents and devices for evaluation of CK levels in a subject, diagnosis of statin-induced myopathy and providing statin therapy. In one embodiment, the invention provides a method for determining an UNL CK level for a subject based on the presence or absence of specific genetic variants. The invention is an application of multiple associations between certain genetic variants and lower or higher on-statin and off-statin CK level in individuals. The invention provides methods for determining an individualized or personalized ULN CK level for a subject, evaluating the subject's on-statin CK level and diagnosing statin-induced myopathy.

In a further embodiment the invention relates to a method of determining a ULN CK level for a subject comprising: (a) genotyping the subject to determine the presence of a genetic variant selected from a G allele of rs142092440, G allele of rs11559024 and G allele of rs12975366, and (b) determining a ULN CK level of 240 U/L if the genetic variant is present.

In another embodiment the invention relates to a method of determining a ULN CK level for a subject comprising: (a) genotyping the subject to determine the presence of a genetic variant selected from a C allele of rs406231 and a T allele of rs2361797, and (b) determining a ULN CK level of 300 U/L if the genetic variant is present.

One embodiment invention provides a method comprising: (a) genotyping a subject for the presence or absence of one or more alleles selected from: a G allele for the SNP rs142092440, a G allele at SNP rs11559024, a G allele at rs12975366, a C allele at rs406231 and a T allele at rs2361797; and (b) determining a ULN CK level for the subject based on the presence or absence of the alleles determined.

In a further embodiment, subjects who carry one or more alleles selected from a G allele at rs142092440, a G allele at rs11559024, a G allele at rs12975366 have a non-pathological on-statin serum CK level between 50 U/L and 90 U/L and a genetically determined ULN CK LEVEL between 150 U/L and 270 U/L.

In a further embodiment, patients who carry on or more alleles selected from a T allele at rs2361797, a C allele at rs406231 have a higher non-pathological on-statin serum CK level between 100 U/L and 120 U/L and a genetically determined ULN CK level between 300 U/L and 360 U/L.

In one embodiment the invention provides a method of evaluating CK level in a subject comprising: genotyping a subject for the presence or absence of one or more genetic variants of a LILRB5 gene, obtaining a measure of the subject's blood CK level and determining a ULN CK level for the subject, based on the presence or absence of the genetic variants analyzed.

In another embodiment the invention provides a method of evaluating a subjects CK level comprising genotyping a subject for the presence or absence of one or more genetic variants of the human CKM gene and one or more genetic variants of the LILRB5 gene and determining a ULN CK level for the subject based on the presence or absence of the genetic variants analyzed. The invention further provides methods of diagnosing or prognosing statin-induced myopathy in a subject comprising: (a) genotyping the presence or absence of one or more of the genetic variants associated listed in Table 2, (b) obtaining a measure of subject's CK level and (c) determining a ULN CK level for a subject wherein the subject is diagnosed with statin-induced myopathy if the CK level obtained in step (b) is greater than the ULN CK level determined in step (c). In an alternative but equivalent embodiment step (b) obtaining a measure of the subject's CK level is performed after step (c) determining a ULN CK level.

In one embodiment of the invention contemplated herein the genotyping step is performed using a genotyping device such as those disclosed in U.S. Patent Applications U.S. 20080275229 and U.S. 20100075296A1. Similarly serum CK levels can be determined using a point-of-care or personal device that can determine CK level from blood sample obtained from for example a finger prick.

The invention provides methods for evaluating of the pathological significance of a subject's blood or serum CK level comprising: determining the presence or absence of two or more minor alleles of SNPs selected from rs142092440, rs11559024, rs12975366, rs406231 and rs2361797 in a subject; and determining an upper limit of normal (ULN) CK level for the subject based on the presence or absence of the two or more genetic variants. A ULN CK level determined based on the presence or absence of genetic variants is also referred to herein as a genetically determined ULN CK level or individualized ULN CK level.

In one embodiment a subject's genetically determined ULN CK level is compared to a measure of the subject's on-statin CK level to determine if the on-statin CK level is indicative of statin-induced myopathy and where the subject's on-statin CK level is more than 3xhigher than their genetically determined ULN CK level, the on-statin CK level is indicative of statin-induced myopathy and statin treatment is terminated.

In one embodiment a subject's genetically determined ULN CK level is compared to a measure of the subject's on-statin CK level to determine if the on-statin CK level is indicative of statin-induced myopathy and where the subject's on-statin CK level is more than 10× higher than their genetically determined ULN CK level, the on-statin CK level is indicative of rhabdomyolysis and statin treatment is terminated.

In one embodiment the invention provides a method for diagnosing statin-induced myopathy in a subject being treated with a statin drug comprising:
a. analyzing the presence or absence of two or more minor alleles of SNPs selected from rs142092440, rs11559024, rs12975366, rs406231, and rs2361797,
b. determining a ULN CK level for the subject based presence or absence of the minor alleles analyzed in step (a);
c. obtaining a measure of blood or serum CK level in the subject and
d. comparing the CK level obtained in step (b) to the ULN CK level determined in step (c) and
e. diagnosing the subject with statin-induced myopathy when the CK level obtained in step (b) is more than 3× greater the ULN CK level determined in step (c).

In the methods contemplated the order of the steps of genotyping, obtaining a measure CK level can be performed in any order either genotyping followed by obtaining a measure of blood or serum CK level or obtaining a measure of blood or serum CK level followed by genotyping.

In one embodiment the invention also provides methods of diagnosing statin-induced myopathy wherein the genotyping methods of the invention further comprises a step of assessing a degree of muscular pain experienced by a subject prior to diagnosing statin-induced myopathy wherein statin-induced myopathy is diagnosed when the degree of muscular pain is above a predetermined pain threshold and the subject's blood or serum CK level is at least 3× greater than the a subjects genetically-determined ULN CK level.

In some embodiments of the invention 2 genetic variants selected from table 2 are genotyped i.e. a minor allele of rs142092440 and minor allele of rs11559024; minor allele of rs142092440 and minor allele of rs12975366; minor allele of rs142092440 and minor allele of rs406231; minor allele of rs142092440 and minor allele of rs2361797; minor allele of rs11559024 and minor allele of rs12975366; minor allele of rs11559024 and minor allele of rs406231; minor allele of rs11559024 and minor allele of rs2361797; minor allele of rs12975366 and minor allele of rs406231; minor allele of rs12975366 and minor allele of rs2361797; or minor allele of rs406231 and minor allele of rs2361797.

In some embodiments of the invention 3 genetic variants selected from table 2 are genotyped i.e. a minor allele of rs142092440, a minor allele of rs11559024 and a minor allele of rs12975366;

a minor allele of rs142092440, a minor allele of rs11559024 and a minor allele of rs406231;

a minor allele of rs142092440, a minor allele of rs11559024 and a minor allele of rs2361797;

a minor allele of rs142092440, a minor allele of rs12975366 and a minor allele of rs406231;

a minor allele of rs142092440, a minor allele of rs12975366 and a minor allele of rs2361797; a

minor allele of rs142092440, a minor allele of rs406231 and a minor allele of rs2361797;

a minor allele of rs11559024, a minor allele of rs12975366 and a minor allele of rs406231;

a minor allele of rs11559024, a minor allele of rs12975366 and a minor allele of rs2361797;

a minor allele of rs11559024, a minor allele of rs406231 and a minor allele of rs2361797; or

a minor allele of rs12975366, a minor allele of rs406231 and a minor allele of rs2361797.

In some embodiments of the invention 4 genetic variants selected from table 2 are genotyped i.e. a minor allele of rs142092440, a minor allele of rs11559024, a minor allele of rs12975366 and a minor allele of rs406231;

a minor allele of rs142092440, a minor allele of rs11559024, a minor allele of rs12975366 and a minor allele of rs2361797;

a minor allele of rs142092440, a minor allele of rs11559024, a minor allele of rs406231 and a minor allele of rs2361797;

a minor allele of rs142092440, a minor allele of rs12975366, a minor allele of rs406231 and a minor allele of rs2361797; or

a minor allele of rs11559024, a minor allele of rs12975366, a minor allele of rs406231 and a minor allele of rs2361797.

In a preferred embodiment of the invention 5 genetic variants selected from table 2 are genotyped i.e. a minor allele of rs142092440, a minor allele of rs11559024, a minor allele of rs12975366, a minor allele of rs406231 and a minor allele of rs2361797.

In one embodiment the invention relates to a method of providing statin therapy to a subject comprising: (a) administering a statin drug to the subject, (b) genotyping the subject to determine the presence of one or more a genetic variants selected from a G allele of rs142092440, a G allele of rs11559024 and a G allele of rs12975366, (c) analyzing a serum sample obtained from the subject to determine an on-statin CK level and (d) continuing statin treatment if the genetic variant is present and the on-statin CK level is lower than 240 U/L.

In another embodiment the invention relates to a method of providing statin therapy to a subject comprising: (a) administering a statin drug to the subject, (b) genotyping the subject to determine the presence of a genetic variant selected from a G allele of rs142092440, a G allele of rs11559024 and a G allele of rs12975366, (c) analyzing a serum sample obtained from the subject to determine an on-statin CK level and (d) terminating statin treatment if the genetic variant is present and the on-statin CK level is greater than 240 U/L.

In a further embodiment, the invention relates to a method of providing statin therapy to a subject comprising: (a) administering a statin drug to the subject, (b) genotyping the subject to determine the presence of a genetic variant selected from a C allele of rs406231 and a T allele of rs2361797, (c) analyzing a serum sample obtained from the subject to determine an on-statin CK level and (d) continuing statin treatment if the genetic variant is present and the on-statin CK level is lower than 300 U/L.

In another embodiment, the invention relates to a method of providing statin therapy to a subject comprising: (a) administering a statin drug to the subject, (b) genotyping the subject to determine the presence of a genetic variant selected from a C allele of rs406231 and a T allele of rs2361797, (c) analyzing a serum sample obtained from the subject to determine CK level and (d) terminating statin treatment if the genetic variant is present and the CK level is greater than 300 U/L.

In another embodiment, the invention relates to a method of treating with a statin a subject having a genome, the method comprising: (i) repeatedly administering the statin to the subject; (ii) genotyping the presence or absence of one or more of the genetic variants listed in Table 2 in the genome; (iii) determining an ULN CK level for the subject based at least in part on the presence or absence of the genetic variants genotyped at step (ii); (iv) obtaining a blood sample from the subject and analyzing the blood sample to measure a sample CK level; and (v) discontinuing administration of the statin to the subject if the sample CK level is above the ULN CK level. Other factors may also be used in determining if the administration of the statin is to be discontinued, for example the factors referred to elsewhere in the present document that are indicative, or that an contribute to a diagnosis, of statin-induced myopathy.

In another embodiment, the invention relates to a method of treating with a statin a subject having a genome, the subject having a personalized ULN CK level determined at least in part from the presence or absence of one or more of the genetic variants listed in Table 2 in the genome, the subject also having a blood CK level, the method comprising: (i) repeatedly administering the statin to the subject; (ii) after step (i), comparing the blood CK level with the personalized ULN CK level; and (iii) discontinuing administration of the statin to the subject if the blood CK level is above the ULN CK level. Other factors may also be used in determining the personalized ULN CK level, for example the factors referred to elsewhere in the present document that are indicative, or that an contribute to a diagnosis, of statin-induced myopathy.

The present invention also provides oligonucleotide detection reagents for use in methods of providing a genetically-determined ULN, methods of diagnosing statin-induced myopathy and methods of providing statin therapy. Such oligonucleotide reagents include primers and probes, genotyping panels, compositions comprising a plurality of reagents for detecting two or more of the genetic variants provided in Table 2 as well as test kits comprising a oligonucleotide detection reagent.

In one embodiment the invention provides a genotyping panel or microarray comprising primers or probes for detecting two or more of the genetic variants listed in Table 2.

The invention provides reagents for detecting a SNP in the context of its flanking nucleotide sequences (which can be either DNA or mRNA) are provided. In particular the reagent can be a hybridization probe or an amplification primer useful for genotype of a SNP of interest.

The invention provides a composition of allele specific probes for detection a set of genetic variants selected from those listed in Table 2 wherein each probe has a length of 15-60 nucleotides and is homologous to a oligonucleotide selected from SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, SEQ. ID. NO. 9, SEQ. ID. NO. 10, SEQ. ID. NO. 11, SEQ. ID. NO. 12, SEQ. ID. NO. 13, SEQ. ID. NO. 14, SEQ. ID. NO. 15, SEQ. ID. NO. 16, SEQ. ID. NO. 17, SEQ. ID. NO. 18, SEQ. ID. NO. 19, or SEQ. ID. NO.20.

The invention provides a composition of primer pairs for PCR-based detection of a set of genetic variants selected from those listed in Table 2 wherein primer has a length of 15-30 nucleotides and is homologous to a oligonucleotide selected from SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, SEQ. ID. NO. 9, SEQ. ID. NO. 10, SEQ. ID. NO. 11, SEQ. ID. NO. 12, SEQ. ID. NO. 13, SEQ. ID. NO. 14, SEQ. ID. NO. 15, SEQ. ID. NO. 16, SEQ. ID. NO. 17, SEQ. ID. NO. 18, SEQ. ID. NO. 19, or SEQ. ID. NO.20.

The invention provides a composition of primers for a primer extension sequencing assay for detection of a set of genetic variants selected from those listed in Table 2 wherein each primer has a length of 15-30 nucleotides and is homologous to a oligonucleotide selected from SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, SEQ. ID. NO. 9, SEQ. ID. NO. 10, SEQ. ID. NO. 11, SEQ. ID. NO. 12, SEQ. ID. NO. 13, SEQ. ID. NO. 14, SEQ. ID. NO. 15, SEQ. ID. NO. 16, SEQ. ID. NO. 17, SEQ. ID. NO. 18, SEQ. ID. NO. 19, or SEQ. ID. NO.20.

The invention provides a composition of oligonucleotide detection reagents, such as primers or probes, for detection of a set of genetic variants selected from those listed in Table 2 wherein the reagents are conjugated to a solid surface.

The invention provides compositions comprising oligonucleotide detection reagents for detection of a set of genetic variants selected from those listed in Table 2 wherein each of reagents is substantially homologous to an oligonucleotide selected from SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, SEQ. ID. NO. 9, SEQ. ID. NO. 10, SEQ. ID. NO. 11, SEQ. ID. NO. 12, SEQ. ID. NO. 13, SEQ. ID. NO. 14, SEQ. ID. NO. 15, SEQ. ID. NO. 16, SEQ. ID. NO. 17, SEQ. ID. NO. 18, SEQ. ID. NO. 19, or SEQ. ID. NO.20 and overlaps a SNP listed in Table 2 having about having about 5, or alternatively 10, or alternatively 20, or alternatively 25, or alternatively 30 nucleotides around the polymorphic region.

The invention includes a test kit for carrying out a method evaluating pathological levels of CK in a subject comprising allele-specific primers or probes. More particularly the invention relates to a test kit for carrying out a method evaluating pathological levels of CK in a subject comprising allele-specific primer or probe for detecting one, two or three of the SNPs listed in Table 2. A test kits of the invention may further comprise, in addition to allele-specific primers or probes, one or more containers containing the detection reagents and one or more components selected from the group consisting of an enzyme, polymerase enzyme, ligase enzyme, buffer, amplification primer pair, dNTPs, ddNTPs, positive control nucleic acid, negative control, nucleic acid extraction reagent, and instructions for using said test kit to determine a pathological CK level or in diagnosing statin-induced myopathy.

All of the above-mentioned aspects and embodiments of the invention recited hereinabove and mentioned herein below may be combined in any suitable manner to obtain more specific embodiments of the invention.

The present application cites a number of documents, the contents of which is hereby incorporated by reference in its entirety.

### BRIEF DESCRIPTION OF THE DRAWINGS

FIG. 1 Manhattan plot showing results of the genome-wide association study for serum CK levels in 3388 statin users showing significant (3 annotated points above p value cut off line indicated) association signals in the CKM (rs11559024), MARK4 (rs56158216) and LILRB5 (rs2361797) gene regions. Each dot represents the −log 10 P value for the genetic association using a multiple regression model adjusted for 2 principal components for genetic ancestry, the case-control myopathy status, age, sex, sampling site, physical activity level and body mass index. The dotted line shows the significance threshold (P=5×10−8).

### DETAILED DESCRIPTION

Definitions

Various features and embodiments of the present invention are disclosed herein; however other features of the invention, modifications and equivalents will be apparent to a person skilled in the relevant art, based on the teachings provided. The invention described is not limited to the examples and embodiments provided, various alternatives equivalents will be appreciate by those skilled in the art.

As used herein, the singular forms “a”, “an” and “the” include the plural unless the context clearly dictates otherwise. For example, “a” cell will also include “cells”.

The term “comprising” is intended to mean that the compositions and methods include the recited elements, but do not exclude others.

“Creatine kinase level” or “CK level” as used herein means a concentration of creatine kinase (CK) in blood or serum of a human subject and any in vivo or in vitro measure thereof. CK level can be determined for example by analyzing the concentration of CK in a serum sample obtained from a subject using standard method well known in the art. In the embodiments of the present invention where CK level is determined CK level can be measured using any method known in the art for measuring CK level. CK level is typically expressed, and expressed herein as a concentration, more specifically as units of CK protein per L of serum (U/L). “Pathological CK level” as used herein means a concentration of CK in blood or serum, measured either in vitro or in vivo, that is associated with statin-induced myopathy in a subject treated with a statin drug. A pathological CK level may be expressed as a range, cut-off or maximum value. The invention is based on the concept that the range of non-pathological or normal CK levels in humans is broader than the currently accepted range of 10 to 150 U/L and that the level of serum CK that is pathological varies between individuals such that currently used ranges of non-pathological levels and cut offs such as the upper limit of normal (ULN) are not widely applicable to a broad population. As a result the threshold values currently used in practice contribute to misdiagnosis of SIM.

“CK upper limit of normal”, “upper limit of normal CK level”, “upper limit of normal”, “ULN CK level” or “ULN” as used herein refers to a cut-off serum CK level where serum CK levels below this cut-off do not indicate the presence of statin-induced myopathy and serum CK levels above this cut-off may indicate the presence of statin-induced myopathy. Evaluation of serum CK level in view of a ULN CK level is used in diagnosing or detecting statin-induced myopathy and muscle damage caused by statins in a patient. Typically statin-induced myopathy is diagnosed when a patient experiences muscle pain symptoms associated with statin-induced myopathy and has an on-statin CK-level greater that 3× the ULN. Diagnosis of more severe forms of statin-induced myopathy is often made when a patient experiences muscle pain symptoms associated with statin-induced myopathy and has an on-statin CK-level greater that 10× the ULN.

“On-statin CK level” as used herein means a CK level determined while a patient taking a statin drug. “Off-statin CK level” as used herein means a CK level determined while a patient is not taking a statin drug.

“Individualized ULN CK level”, “personalized ULN CK level”, “Individualized ULN” or “Personalized ULN”, as used herein means a ULN CK level determined for a particular patient based on (i) genotype information obtained from the patient or (ii)a combination of genotype information obtained from the patient, and combination with other known clinical risk factors associated with statin induced myopathy or CK level. Importantly the methods of the invention can be used to determine an individualized or personalized ULN CK level for a patient. Serum levels of CK below a personalized or individualized ULN are considered non-pathological and not indicative of statin-induced myopathy and serum levels of CK above the ULN are considered pathological and indicative of statin-induced myopathy.

Individualized or personalized as used herein means clinically relevant information, diagnostic information, a diagnosis, a prognosis or a therapeutic approach that is tailored to an individual patient, according to specific genomic, genetic or phenotypic characteristics of the individual.

A “gene” is an ordered sequence of nucleotides located in a particular position on a particular chromosome that encodes a specific functional product and may include un-translated and un-transcribed sequences in proximity to the coding regions. Such non-coding sequences may contain regulatory sequences needed for transcription and translation of the sequence or introns etc. or may as yet to have any function attributed to them beyond the occurrence of the SNP of interest.

An “allele” is defined as any one or more alternative forms of a given gene. In a diploid cell or organism the members of an allelic pair (i.e. the two alleles of a given gene) occupy corresponding positions (loci) on a pair of homologous chromosomes and if these alleles are genetically identical the cell or organism is said to be “homozygous”, but if genetically different the cell or organism is said to be “heterozygous” with respect to the particular gene.

“CKM gene” or “CKM” as used herein means the Homo sapiens creatine kinase, muscle gene NCBI Gene ID: 1158. Related sequences included ENSG00000104879; HPRD:00426; MIM:123310; Vega:OTTHUMG00000181782. Other names for the CKM gene include CKMM; M-CK. The protein encoded by this gene is a cytoplasmic enzyme involved in energy homeostasis and is an important serum marker for myocardial infarction. The encoded protein reversibly catalyzes the transfer of phosphate between ATP and various phosphogens such as creatine phosphate. It acts as a homodimer in striated muscle as well as in other tissues, and as a heterodimer with a similar brain isozyme in heart. The encoded protein is a member of the ATP:guanido phosphotransferase protein family.

“LILRB5 gene” or “LILRB5” as used herein means the Homo sapiens leukocyte immunoglobulin-like receptor, subfamily B (with TM and ITIM domains), member 5, NCBI Gene ID: 10990. Related sequences include Ensembl:ENSG00000105609; HPRD:11993; MIM:604814; Vega:OTTHUMG00000066636. The LILRB5 gene is also known as LIRE; CD85C; LIR-8. LILRB5 is a member of the leukocyte immunoglobulin-like receptor (LIR) family, which is found in a gene cluster at chromosomal region 19q13.4. The encoded protein belongs to the subfamily B class of LIR receptors which contain two or four extracellular immunoglobulin domains, a trans-membrane domain, and two to four cytoplasmic immune-receptor tyrosine-based inhibitory motifs (ITIMs). Several other LIR subfamily B receptors are expressed on immune cells where they bind to MHC class I molecules on antigen-presenting cells and inhibit stimulation of an immune response. Multiple transcript variants encoding different isoforms have been found for this gene. As used herein LILRB5 gene refers to both the coding and non-coding regions of the LILRB5 gene.

“Genotyping” refers to the determination of the genetic information an individual carries at one or more positions in the genome. For example, genotyping may comprise the determination of which allele or alleles an individual carries for a single SNP or the determination of which allele or alleles an individual carries for a plurality of SNPs. For example, at rs406231 the nucleotide at this position may be a C (cytosine) in some individuals and an A (adenine) in other individuals. Individuals who have or carry a C at the position indicated by rs406231 have the C allele. Individuals who have or carry an A at the position indicated by rs406231 have the A allele. In a diploid organism an individual will have two copies of the sequence containing the polymorphic position so the individual may have an A allele and a C allele or alternatively, two copies of the A alleles or two copies of the C allele. Those individuals who have two copies of the C allele are homozygous for the C allele, those individuals who have two copies of the A allele are homozygous for the A allele, and those individuals who have one copy of each allele are heterozygous.

The two copies of each alleles in a diploid organism can be referred to as the major allele (A) and the minor allele (B) and genotypes represented as AA (homozygous A), BB (homozygous B) or AB (heterozygous). Genotyping methods generally provide for identification of the sample as AA, BB or AB. Minor allele refers to the nucleotide found less frequently in a given population (i.e. Tat rs2361797). Major allele refers to the nucleotide found more frequently in a given population (i.e. C at rs2361797).

The term “polymorphism” “polymorphism site” “polymorphic site” or “single nucleotide polymorphism site” (SNP site) or “single nucleotide polymorphism” refers to a location in the sequence of a gene which varies within a population. A polymorphism is the occurrence of two or more forms of a gene or position within a gene allele, in a population, in such frequencies that the presence of the rarest of the forms cannot be explained by mutation alone. Preferred polymorphic sites have at least two alleles. The implication is that polymorphic alleles confer some phenotype variability on the host. Polymorphisms, SNPs, genetic variants occur in both coding regions and noncoding regions of genes. Polymorphism may occur at a single nucleotide site or may involve an insertion or a deletion. The location of a polymorphism may be identified by its nucleotide position in: a gene, a chromosome or amino acid transcript corresponding to a nucleotide polymorphism. Individual polymorphisms are assigned unique identifiers (“Reference SNP”, “refSNP” or “rs#”). These identifiers are known to one of skill in the art and generally used to refer to and name a polymorphic site, for example identifiers provided in the NCBI Single Nucleotide Polymorphism Database (dbSNP). An identifier with an “rs” prefix, e.g. rs142092440 refers to a SNP included in the dbSNP database (http://www.ncbi.nlm.nih.gov/snp/?term). The “rs” numbers are the NCBI rsSNP ID form.

“Genetic variant” as used herein means DNA sequence variation that occurs in a population. For example a minor allele of a given SNP that is associated with blood or serum CK levels in a human, such as those listed in Table 2. Specific genetic variants are referred to herein as a nucleotide present at a position indicated by an “rs” number. For example “G at rs406231” or “G of rs406231” means that the genetic variant being referred to is a guanine nucleotide present at the SNP site rs406231. Genetic variants genotyped in the methods of the invention include:
a G (guanine) allele at rs142092440 where the sense strand (+) is detected,
a C (guanine) allele at rs142092440 where the anti-sense strand (−) is detected,
a C (cytosine) allele at rs11559024 where the sense strand (+) is detected,
a G (guanine) allele at rs11559024 where the anti-sense strand (−) is detected,
a C (cytosine) allele at rs12975366 where the sense strand (+) is detected,
a G (guanine) allele at rs12975366 where the anti-sense strand (−) is detected,
a G (guanine) allele at rs406231 where the sense strand (+) is detected,
a C (cytosine) allele at rs406231 where the anti-sense strand (−) is detected,
an A (adenine) at rs2361797 where the sense strand (+) is detected, and
an T (thymine) at rs2361797 where the anti-sense strand (−) is detected.

These genetic variants are referred to according to the minor allele nucleotide present in the sense or anti-sense strand as indicated. In the methods of the invention, genetic variants associated with CK level, minor alleles of rs142092440, rs11559024, rs12975366, rs406231 or rs2361797, can be determined based on analysis of a DNA sense (+) or antisense (−) strand. Probes or primers can be designed to bind to either a sense or antisense strand comprising a specific genetic variant, in particular those listed in Table 2.

The terms “polynucleotide”, “nucleotide”, “nucleotide sequence”, refer nucleic acid polymers of any length. DNA or RNA polymers can be either synthetic (synthesized in vitro) or genomic (synthesized in vivo), single or double stranded, may comprise non-naturally occurring DNA or RNA monomers and may comprise any modified, labeled, or conjugated form of a DNA or RNA monomer. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, oligonucleotides, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Oligonucleotides” as used herein means a polynucleotide of less than 200 nucleotides. Synthetic (synthesized in vitro) oligonucleotides are useful as probes, primers which can be used in a variety of ways as reagents for detecting a genetic variant. An oligonucleotide is generally comprised of a single stranded polynucleotide strand of 200 base pairs. Manufactured or synthetic oligonucleotides, useful as primers or probes, are used in genotyping assays under conditions that allow for high specificity hybridization of a single stranded primer or probe with single stranded genomic polynucleotide comprising a genetic variant or variants of interest forming a hybrid double stranded polynucleotide comprising both the synthetic reagent and genomic polynucleotide. The present invention relates in particular to synthetic oligonucleotide “primers”, “primer pairs” or probes that can be used as reagents to genotype the genetic variants listed in Table 2. Primers or probes are 12 to 80 bases in length and preferably 15, 16, 17, 18, 19, 20, 21, 22, 23, 24, 25, 26, 27, 28, 29, 30, 31, 32, 33, 34, 35, 36, 37, 38, 39 or 40 bases in length.

More particularly as used herein “primer” refers a short oligonucleotide, generally with a free 3′-OH group, that binds to a target polynucleotide or “template” potentially present in a sample of interest by hybridizing with the target, and thereafter promoting polymerization of a polynucleotide complementary to the target. “CKM primer” refers to a primer suitable for hybridization of a CKM target polynucleotide. “LILRB5 primer” refers to a primer suitable for hybridization of a LILRB5 target polynucleotide. The terms “primer”, “probe” or “oligonucleotide reagent” includes chemically synthesized single stranded oligonucleotides as used herein these terms do not refer to naturally occurring single stranded oligonucleotides.

A “nucleotide probe” or “probe” refers to an oligonucleotide reagent used for detecting or identifying a target polynucleotide in a hybridization reaction. The term “probes” refers to synthetic oligonucleotides chemically synthesized in vitro (man-made), single stranded nucleic acids designed and manufactured as a reagent for detecting the presence or absence of a particular genetic variant in a genomic (naturally occurring) DNA sample.

A “control” is an alternative subject or sample used in an experiment for comparison purpose. A control can be “positive” or “negative”. For example, where the purpose of the experiment is to determine a correlation of an altered expression level of a gene with atherosclerosis or atherogenesis, it is generally preferable to use a positive control (a subject or a sample from a subject, carrying such alteration and exhibiting syndromes characteristic of atherosclerosis or atherogenesis), and a negative control (a subject or a sample from a subject lacking the altered expression and syndromes characteristic of atherosclerosis or atherogenesis).

An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

The term “sample” includes any biological sample taken from a human patient, individual or subject including a cell, tissue sample or bodily fluid. For example, a sample may include blood, saliva, buccal cells, biopsy sample, sinovial fluid or cerebrospinal fluid. A sample can include, without limitation, an aliquot of a body fluid, whole blood, platelets, serum, plasma, red blood cells, white blood cells, saliva, endothelial cells, tissue biopsies, synovial fluid and lymphatic fluid. Preferably the sample for use in the present invention, in determining circulating CK level, is a blood sample. For determining a blood CK level a blood sample is typically processed to provide serum and soluble CK is measured. This measure of CK level corresponds to the soluble fraction of CK present in the blood sample collected. Samples of particular use in obtaining a polynucleotide sample from a subject are a blood sample, saliva sample of buccal cells. Preferably the sample for use in the genotyping methods of the invention is a saliva sample, alternatively a blood sample.

“Stringent hybridization conditions”, “high stringency conditions” or “high stringency hybridization” as used herein means hybridizing at 68° C. in 5×SSC/5× Denhardt's solution/1.0% SDS, and washing in 0.2×SSC/0.1% SDS at room temperature, or involve the art-recognized equivalent thereof. Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al. “Molecular Cloning: A Laboratory Manual”, 4th Edition, (2012) and F. Ausubel et al, eds., “Current protocols in molecular biology” Chapter 2, Wiley Interscience, (2012). Stringent hybridization conditions allow for specific binding of single stranded complimentary polynucleotide sequences while minimizing non-specific binding between non-complimentary sequences.

The term “subject” includes, without limitation, humans and non-human primates, livestock animals, companion animals, laboratory test animals, captive wild animals, reptiles and amphibians, fish, birds and any other organism. The most preferred subject of the present invention is a human. A subject, regardless of whether it is a human or non-human organism may be referred to as a patient, individual, subject, animal, host or recipient. Subject, patient and individual are use interchangeably herein.

In general a “substantially homologous nucleotides” or “substantially homologous oligonucleotides” are at least about 80% identical with each other, after alignment of the homologous regions. Preferably, the sequences are at least about 85% identical; more preferably, they are at least about 90% identical; more preferably, they are at least about 90% identical; still more preferably, the sequences are more than 95% identical. Sequence alignment and homology searches can be determined with the aid of computer methods. A variety of software programs are available in the art. Non-limiting examples of these programs are Blast, Fasta (Genetics Computing Group package, Madison, Wis.), DNA Star, MegAlign, Tera-BLAST (Timelogic) and GeneJocky. Any sequence databases that contains DNA sequences corresponding to a target gene or a segment thereof can be used for sequence analysis. Commonly employed databases include but are not limited to GenBank, EMBL, DDBJ, PDB, SWISS-PROT, EST, STS, GSS, and HTGS. Common parameters for determining the extent of homology set forth by one or more of the aforementioned alignment programs include p value and percent sequence identity. P value is the probability that the alignment is produced by chance. For a single alignment, the p value can be calculated according to Karlin et al. (1990) Proc. Natl. Acad. Sci 87: 2246. For multiple alignments, the p value can be calculated using a heuristic approach such as the one programmed in Blast. Percent sequence identity is defined by the ratio of the number of nucleotide matches between the query sequence and the known sequence when the two are optimally aligned. To determine that nucleotide sequences are substantially homologous, it is useful to first establish the lowest temperature at which only homologous hybridization occurs with a particular concentration of salt (e.g., SSC or SSPE).

Then, assuming that 1% mismatching results in a 1° C. decrease in the Tm, the temperature of the final wash in the hybridization reaction is reduced accordingly (for example, if sequences having >95% identity are sought, the final wash temperature is decreased by 5° C.). In practice, the change in Tm can be between 0.5° C. and 1.5° C. per 1% mismatch.

Genetic Variants of the Invention

Genetic variants determined in the methods of the invention and detected by the compositions, reagents and of the invention are single nucleotide polymorphisms (SNPs) of the human CKM or LILRB5 gene and more particularly the specific genetic variants of shown in Table 2.

TABLE 2
Genetic Variants Associated with CK Level
TABLE 3

| | | | Minor/ | |
|---|---|---|---|---|
| | | | Major | |
| | | | Allele | Allele - CK level |
| Gene | Position | RS number | (strand) | association |
| CKM | Chr19: 45810010 | rs142092440 | G/A (+) | G - lower CK level |
| CKM | Chr19: 45810010 | rs142092440 | C/T (−) | C - lower CK level |
| CKM | Chr19: 45821183 | rs11559024 | C/T (+) | C - lower CK level |
| CKM | Chr19: 45821183 | rs11559024 | G/A (−) | G - lower CK level |
| LILRB5 | Chr19: 54759361 | rs12975366 | C/T (+) | C- lower CK level |
| LILRB5 | Chr19: 54759361 | rs12975366 | G/A (−) | G- lower CK level |
| LILRB5 | Chr19: 54753542 | rs406231 | G/T (+) | G - higher CK level |
| LILRB5 | Chr19: 54753542 | rs406231 | C/A (−) | C - higher CK level |
| LILRB5 | Chr19: 54751064 | rs2361797 | A/G (+) | A - higher CK level |
| LILRB5 | Chr19: 54751064 | rs2361797 | T/C (−) | T - higher CK level |

In Table 2 and 3 (+) and (−) indicate positive or negative DNA strand corresponding to the listed minor/major alleles . The genetic variants, minor alleles, listed can be detected by analyzing the sense or antisense strand or a combination thereof depending on the binding specificity of the probes or primers used in the assay. In the column “Allele −CK level association”, “lower CK level” indicates that the minor allele indicated in the row is associated with lower CK levels compared to individuals who carry the major allele and “higher CK level” indicates that the minor allele indicated in the row is associated with higher CK levels compared to individuals who carry the major allele.

TABLE 3
Oligonucleotides Comprising Genetic
Variants of the Invention (nucleotide
corresponding to allele of interest indicated
in larger font and underlined)

| SEQ ID | | SNP | | |
|---|---|---|---|---|
| NO | SNP site | allele | Strand | Sequence |
| SEQ. ID. | rs142092440 | G | (+) | CCCTCCCACTGGCTGGGTTCCAGCAGTCGGTGGCA |
| NO. 1 | | | | GGTGGGCAGGCGCCT [symbol] CTTCTGGGCGGGGATCAT |
| | | | | GTCGTCAATGGACTGGCCTTTCTCCAACTTCT |
| SEQ. ID. | rs142092440 | A | (+) | CCCTCCCACTGGCTGGGTTCCAGCAGTCGGTGGCA |
| NO. 2 | | | | GGTGGGCAGGCGCCT [symbol] CTTCTGGGCGGGGATCAT |
| | | | | GTCGTCAATGGACTGGCCTTTCTCCAACTTCT |
| SEQ. ID. | rs142092440 | C | (-) | AGAAGTTGGAGAAAGGCCAGTCCATTGACGACATG |
| NO. 3 | | | | ATCCCCGCCCAGAAG [symbol] AGGCGCCTGCCCACCTGCC |
| | | | | ACCGACTGCTGGAACCCAGCCAGTGGGAGGG |
| SEQ. ID. | rs142092440 | T | (-) | AGAAGTTGGAGAAAGGCCAGTCCATTGACGACATG |
| NO. 4 | | | | ATCCCCGCCCAGAAG [symbol] AGGCGCCTGCCCACCTGCC |
| | | | | ACCGACTGCTGGAACCCAGCCAGTGGGAGGG |
| SEQ. ID. | rs11559024 | T | (+) | AGCCCCCGTGGCGATCCGAGATGATGGGGTCAAAG |
| NO. 5 | | | | AGTTCCTTGAAAACT [symbol] CGTAGGACTCCTCATCACCA |
| | | | | GCCACGCAGCCCACGGTCATGATGAAGGGG |
| SEQ. ID. | rs11559024 | C | (+) | AGCCCCCGTGGCGATCCGAGATGATGGGGTCAAAG |
| NO. 6 | | | | AGTTCCTTGAAAACT [symbol] CGTAGGACTCCTCATCACCA |
| | | | | GCCACGCAGCCCACGGTCATGATGAAGGGG |
| SEQ. ID. | rs11559024 | A | (-) | CCCCTTCATCATGACCGTGGGCTGCGTGGCTGGTG |
| NO. 7 | | | | ATGAGGAGTCCTACG [symbol] AGTTTTCAAGGAACTCTTT |
| | | | | GACCCCATCATCTCGGATCGCCACGGGGGCT |
| SEQ. ID. | rs11559024 | G | (-) | CCCCTTCATCATGACCGTGGGCTGCGTGGCTGGTG |
| NO. 8 | | | | ATGAGGAGTCCTACG [symbol] AGTTTTCAAGGAACTCTTT |
| | | | | GACCCCATCATCTCGGATCGCCACGGGGGCT |
| SEQ. ID. | rs12975366 | C | (+) | CGAGGTCATGTTCCCCCTCCTTGTACAGAACGAATA |
| NO. 9 | | | | TGTCATAGCCGACA [symbol] CAGAGCGACACTGCAGGGTC |
| | | | | AGGCTGCCTCCGCGGGCCACGACAGAGCCC |
| SEQ. ID. | rs12975366 | T | (+) | CGAGGTCATGTTCCCCCTCCTTGTACAGAACGAATA |
| NO. 10 | | | | TGTCATAGCCGACA [symbol] CAGAGCGACACTGCAGGGTC |
| | | | | AGGCTGCCTCCGCGGGCCACGACAGAGCCC |
| SEQ. ID. | rs12975366 | G | (-) | GGGCTCTGTCGTGGCCCGCGGAGGCAGCCTGACCC |
| NO. 11 | | | | TGCAGTGTCGCTCTG [symbol] TGTCGGCTATGACATATTC |
| | | | | GTTCTGTACAAGGAGGGGGAACATGACCTCG |
| SEQ. ID. | rs12975366 | A | (-) | GGGCTCTGTCGTGGCCCGCGGAGGCAGCCTGACCC |
| NO. 12 | | | | TGCAGTGTCGCTCTG [symbol] TGTCGGCTATGACATATTC |
| | | | | GTTCTGTACAAGGAGGGGGAACATGACCTCG |
| SEQ. ID. | rs406231 | T | (+) | CTTTGGTTGGTGCCCTGATCCCACCCTCGGTGGGCC |
| NO. 13 | | | | CACAGGTTCCCCCA [symbol] TCCCTGCTCACCCAATGTCCT |
| | | | | GTGTTTGCTCTGACGCCGACATTGGAGGA |
| SEQ. ID. | rs406231 | G | (+) | CTTTGGTTGGTGCCCTGATCCCACCCTCGGTGGGCC |
| NO. 14 | | | | CACAGGTTCCCCCA [symbol] TCCCTGCTCACCCAATGTCCT |
| | | | | GTGTTTGCTCTGACGCCGACATTGGAGGA |
| SEQ. ID. | rs406231 | A | (-) | TCCTCCAATGTCGGCGTCAGAGCAAACACAGGACA |
| NO. 15 | | | | TTGGGTGAGCAGGGA [symbol] TGGGGGAACCTGTGGGCC |
| | | | | CACCGAGGGTGGGATCAGGGCACCAACCAAAG |
| SEQ. ID. | rs406231 | C | (-) | TCCTCCAATGTCGGCGTCAGAGCAAACACAGGACA |
| NO. 16 | | | | TTGGGTGAGCAGGGA [symbol] TGGGGGAACCTGTGGGCC |
| | | | | CACCGAGGGTGGGATCAGGGCACCAACCAAAG |
| SEQ. ID. | rs2361797 | G | (+) | AGGAAGAGAAAACGATGTCTAGCAATAGCCCAAGA |
| NO. 17 | | | | GGTGAGTAGCTGAAC [symbol] TTTTATAGAGATGAGGAG |
| | | | | AGACTAACTAAGGACTAGGGCGCATCCCTTTA |
| SEQ. ID. | rs2361797 | A | (+) | AGGAAGAGAAAACGATGTCTAGCAATAGCCCAAGA |
| NO. 18 | | | | GGTGAGTAGCTGAAC [symbol] TTTTATAGAGATGAGGAG |
| | | | | AGACTAACTAAGGACTAGGGCGCATCCCTTTA |
| SEQ. ID. | rs2361797 | C | (-) | TAAAGGGATGCGCCCTAGTCCTTAGTTAGTCTCTCC |
| NO. 19 | | | | TCATCTCTATAAAA [symbol] GTTCAGCTACTCACCTCTTGG |
| | | | | GCTATTGCTAGACATCGTTTTCTCTTCCT |
| SEQ. ID. | rs2361797 | T | (-) | TAAAGGGATGCGCCCTAGTCCTTAGTTAGTCTCTCC |
| NO. 20 | | | | TCATCTCTATAAAA [symbol] GTTCAGCTACTCACCTCTTGG |
| | | | | GCTATTGCTAGACATCGTTTTCTCTTCCT |

As indicated in Table 2 the alleles listed are associated with lower or higher circulating CK levels in subjects taking a statin drug (on-statin CK level) or subjects not taking a statin drug (off-statin CK level). Subjects homozygous or heterozygous for one or more minor alleles of a SNP associated with low CK level, i.e. rs142092440, rs11559024 or rs12975366, have lower off-statin and on-statin CK levels and a lower genetically determined ULN CK level.

Subjects homozygous or heterozygous for one or more minor alleles of a SNP associated with high CK level, i.e. rs406231 and rs2361797, have higher off-statin and on-statin CK levels compared to non-carriers or subject who carry a minor allele of rs142092440, rs11559024 or rs12975366. CK levels indicative of statin-myopathy are lower in subjects who carry a minor allele of rs142092440, rs11559024 or rs12975366 compared to non-carriers and compared to subjects who carry a genetic variant associated with high CK level e.g. C allele of rs406231 or A allele of rs2361797. CK levels indicative of statin-myopathy are higher in subjects who carry one or more minor alleles of rs406231 or rs2361797 compared to non-carriers or carriers of genetic variants associated with low CK level e.g. G allele of rs142092440 and the G allele of rs11559024.

The ULN CK level, pathological or non-pathological CK level range determined for a subject using the methods of the present invention is used in combination with other factors to diagnose or prognose statin-induced myopathy in the subject. For example if the ULN CK level determined for a patient is >150 U/L and the on-statin serum CK level for the patient is 200 U/L then the serum CK level indicates the presence of statin-induced myopathy. If the ULN CK level determined for a patient is >300 U/L and the statin CK level and the CK level is 200 U/L then the serum CK level does not indicate the presence of statin-induced myopathy. In contrast to currently used methods of evaluating CK level in diagnosing statin-induced myopathy, pathological levels are determined for each individual based on the individual's genotype or a combination of genotype and other known risk factors for statin-induced myopathy including but not limited to age, sex, physical activity or exercise. A pathological range, level or cut-off or ULN for a subject can be determined using the methods of the invention either prior to or following initiation of statin treatment i.e. on-statin or off-statin. Additionally measures of the subjects off-statin CK level can also be considered with genotype information to determine pathological range, level or cut-off or ULN for the subject.

Genotyping genetic variants of the CKM gene, LILRB5 gene or the CKM gene and LILRB5 gene is useful in determining a CK level in an individual that is pathological or indicative of statin-induced myopathy when the individual is administered a statin drug. Genotyping the SNPs in Table 2 and determining a ULN CK level for a subject is useful prior to or during statin administration and provides an improve method for diagnosing statin-induced myopathy.

In some embodiments of the methods of the invention the genotyping step, analyzing the presence or absence of an allele is performed by querying pre-existing data obtained from a prior analysis of a DNA sample obtained from the subject. In other embodiments the genotyping step comprises obtaining a biological sample from a subject and analyzing the sample to determine a genotype or genotypes, using any suitable method known in the art for detecting the presence or absence of a specific allele.

In the methods of the invention analyzing or assaying a sample to determine a genotype may comprise detecting an amplified polynucleotide, which is produced by amplifying nucleic acid template comprising a genetic variant of interest, including but not limited to those provided in Table 2.

In some embodiments the methods of the invention comprises the step of “obtaining a measure of the subjects blood CK levels” this step may comprise obtaining a blood sample from the subject and determining a CK level using standard methods or may comprise querying pre-existing data to obtain a measure of the subjects blood CK level derived from a previously performed analysis. To determine CK level a blood sample is processed to provide a serum sample and a CK concentration is determined in the serum sample. The serum CK concentration corresponds to the soluble blood CK concentration.

In a further embodiment the genotyping panel or microarray also comprises primers or probes for detection other genetic variants associated with statin response, or a statin-induced side effect including statin-induced myopathy.

Numerous such genetic variants are known in the art including but not limited to rs4693596 of the COQ2 gene (Oh J et al. Lipids Health Dis 6:7 2007). In particular the invention relates to genetic variants of the human CKM and LILRB5 genes and their use in determining non-pathological or pathological blood CK levels in an individual, where pathological means associated with or contributing to statin-induced myopathy. In one embodiment the method includes assaying the presence or absence one or more variants of the CKM or LILRB5 gene and determining a ULN CK level for the subject. In a further embodiment, the method includes the steps of analyzing the presence or absence of one or more minor alleles of a SNP selected from: rs142092440, rs11559024, rs12975366, rs406231 and rs2361797, obtaining a measure of the subject's serum or blood CK level and determining a pathological blood CK level, range or cut-off for the subject based on the presence or absence of one or more of the alleles analyzed. The presence of one or more of the minor alleles of the SNPs in Table 2 in a subject's genome is associated with a higher or lower CK level compared to non-carriers. A G allele at rs406231 and a A allele at rs2361797 are associated with a higher on-statin or off-statin CK-level. The G allele at SNP rs142092440, G allele at rs11559024, and C allele at rs12975366 are associated with lower on-statin or off statin CK-levels.

Genetic variants for use in the present invention include those included in Table 2, any genetic variant of the CKM or LILRb5 gene associated with lower or higher on-statin or off-statin CK level.

The effects of the presence of genetic variants associated with CK level, such as those in Table 2, can be additive for example, the ULN CK level is relatively low e.g. between 100 U/L to 200 U/L (expressed as units/liter of serum) in homozygous carriers or carriers of multiple variants associated with lower serum CK levels. Alternatively it is contemplated that genetic variants associated with higher on-statin CK levels are also useful in the methods of the present invention. For example pathological blood CK levels may be very high e.g. 500-700 U/L in carriers of variants associated with high on-statin CK level. Furthermore the effect of the presence of a genetic variant associated with lower CK levels may be mitigated or neutralized when the individual also carries a variant associated higher CK level. As new associations between on-statin CK level and genetic variants are identified and validated it will be clear to a person skilled in the how to apply these associations as part of the methods for evaluating CK levels in a subject disclosed herein.

The presence or absence of one or more genetic variants in the CKM gene or LILRB5 gene, and particularly the genetic variants listed in Table 2, can be considered in combination with other factors known to influence CK level in subjects on statin. Factors known to be associated with on-statin CK levels include sex, age, concomitant drug use and degree of physical activity. In yet a further embodiment the presence or absence of one or more of the genetic variants in Table 2 are considered in combination with other factors known to influence blood CK levels and the subject's pathological CK level is determined based on a combination of genotype and other factors including but not limited to sex, age, concomitant drug use and degree of physical activity.

The methods and SNPs disclosed herein are also useful in the development and validation of therapeutic agents. Method of selecting patients for inclusion in a clinical trial of a statin therapy (e.g. selecting individuals for participation in a clinical trial that are least likely to experience elevated CK levels during statin treatment or excluding individuals

The genotyping methods of the invention are useful in selecting or formulating a statin treatment regimen such as dosage, frequency of administration or a particular form/type of statin. In one embodiment the invention includes a method of stratifying a patient population for treatment with a statin drug. This stratification method includes evaluating the likelihood that subjects treated with a statin drug will experience statin-induced myopathy based on the subjects' genotype or based on a combination of genotype information and other risk factors known to be associated with risk of statin-induced myopathy. Methods, assays, reagents and kits for detecting the presence of the polymorphisms listed in Table and their encoded products are provided.

Currently the upper limit of normal (ULN) CK level in females is thought to be between 10 and 79 units per liter of serum (U/L) and the ULN CK level in males is thought to be between 17 and 148 U/L. The present invention is based on the concept that the range of healthy or normal CK levels in males and females is broader and more diverse than currently accepted normal levels. Further the invention is based on the finding that normal or pathological levels in individuals are genetically determined and certain genetic variants can be used to determine a normal range and ULN CK threshold for an individual. Accordingly the present invention provides genetic variants, methods, reagents and kits for evaluating a subjects CK level and determining an individualized ULN CK threshold

Patient-related risk factors for statin-related myotoxicity include female gender, low body mass index, concomitant treatment with certain cytochrome P450 inhibitors, a decline in renal and hepatic function, and changes in albumin and a-1 glycoprotein levels with subsequent changes in free concentration levels of statins (Jacobson, T. A., 2008 Mayo Clinic Proceedings 83, 687-700. doi:10.4065/83.6.687). Statin myopathy is dose-related. An increase in statin dose and statin systemic exposure magnifies the risk of muscle toxicity ((Jacobson, T. A., 2008 Mayo Clinic Proceedings 83, 687-700. doi:10.4065/83.6.687). A genome-wide study has identified common genetic variants in SLCO1B1 that are associated with substantial alterations in the risk of simvastatin-induced myopathy (Link, E. et al. 2008 NEJM 359, 789-799. doi:10.1056/NEJMoa0801936). The finding of an association between SLCO1B1-rs4149056 and statin-induced myotoxicity has since been replicated in both an independent trial and a practice-based longitudinal cohort (Voora D. and Ginsberg G S. J. Am. Coll. Cardiol. 60, 9-20. doi:10.1016/j.jacc.2012.01.067, 2009; Bulbulia, R. et al., 2011 The Lancet 378, 2013-2020. doi:10.1016/50140-6736(11)61125-2). Recently, the Clinical Pharmacogenomics Implementation Consortium published a guideline paper that discusses the relationship between rs4149056 and clinical outcome for simvastatin (Wilke, R. A. et al. 2012 Clinical Pharmacology & Therapeutics 92, 112-117. doi:10.1038/clpt.2012.57).

Any factor the increases the serum concentration of a statin increases an individual's risk of statin-induced myopathy such as pharmacodynamic factors that affect the transport, metabolism or bioavailability of statin drugs. Other factors associated with an increased risk of statin-induced myopathy include: alcohol consumption; heavy exercise; surgery with severe metabolic demands; drugs affecting the cytochrome P450 system; cyclosporine; fibrates; nicotinic acid; nondihydropyridine calcium channel blockers eg, verapamil (calan), diltiazem (cardizem); amiodarone (cordarone); azole antifungals; colchicine; digoxin; human immunodeficiency virus protease inhibitors; warfarin (coumadin); and consuming >1 l of grapefruit juice per day. These factors can be used in methods of the invention in combination with genotype information to determine a pathological CK level of an individual or to diagnose statin-induced myopathy.

Endogenous factors associated with an increased risk of statin-induced myopathy include: Advanced age (>65 years); Low body mass index and frailty; Multisystem disease; Renal dysfunction; Hepatic dysfunction; Thyroid disorders, especially hypothyroidism; Hypertriglyceridemia; Metabolic muscle diseases;Carnitine palmityl transferase II deficiency; McArdle disease (myophosphorylase deficiency); Myoadenylate deaminase deficiency; Family history of muscular symptoms and Personal history of elevated creatine kinase or muscular symptoms. Such endogenous factors can be used in methods of the invention in combination with genotype information to determine a pathological CK level of an individual or to diagnose statin-induced myopathy.

One or more associated endogenous or exogenous factors can be used in combination with the methods of the present inventor to prognose or diagnose statin-induced myopathy.

The propensity of lipophilic statins (simvastatin, atorvastatin, lovastatin) to induce myopathy is higher than for hydrophilic statins (prevastatin, rosuvastatin, and fluvastatin). Lipophilic compounds are more likely to penetrate the muscle and cause myotoxic effects (Thompson P D et. al. 2003 JAMA 289, 1681. doi:10.1001/jama.289.13.1681).

Algorithmic approaches for diagnosing statin-induced myopathy based on a combination of exogenous and endogenous factors are known in the art (Fernandez G. et al. 2011 Cleveland Clinic Journal of Medicine 78, 393-403. doi:10.3949/ccjm.78a.10073; Sai K, et al. 2013 Journal of Clinical Pharmacy and Therapeutics 38, 230-235. doi:10.1111/jcpt.12063; Venero C V et al. 2009 Endocrinology and Metabolism Clinics of North America 38, 121-136. doi:10.1016/j.ec1.2008.11.002, Rallidis L S et al. 2012 International Journal of Cardiology 159, 169-176. doi:10.1016/j.ijcard.2011.07.048). These approaches, and any other suitable ones, can be used in the present invention.

In some embodiments the invention provides methods of providing statin therapy or methods of treatment. In one embodiment of the invention, an individualized ULN CK level is determined for a subject taking a statin based on genotyping one or more of the genetic variants listed in Table 2, serum CK level is determined for the subject, the serum CK level is found to be higher than the ULN CK level determined and administration of the statin is terminated. In another embodiment an individualized ULN CK level is determined for a subject taking a statin based on genotyping one or more of the genetic variants listed in Table 2, serum CK level is determined for the subject, the serum CK level is found to be lower than the ULN CK level determined and administration of the statin is continued.

Genotyping Methods

Identification of the particular genotype in a sample may be performed by any of a number of methods well known to one of skill in the art. For example, identification of the polymorphism can be accomplished by cloning of the allele and sequencing it using techniques well known in the art. Alternatively, the gene sequences can be amplified from genomic DNA, e.g. using PCR, and the product sequenced. Numerous methods are known in the art for isolating and analyzing a subject's DNA for a given genetic marker including polymerase chain reaction (PCR), ligation chain reaction (LCR) or ligation amplification and amplification methods such as self-sustained sequence replication. Several non-limiting methods for analyzing a patient's DNA for mutations at a given genetic locus are described below. DNA microarray technology, e.g., DNA chip devices and high-density microarrays for high-throughput screening applications and lower-density microarrays, may be used. Methods for microarray fabrication are known in the art and include various inkjet and microjet deposition or spotting technologies and processes, in situ or on-chip photolithographic oligonucleotide synthesis processes, and electronic DNA probe addressing processes. The DNA microarray hybridization applications has been successfully applied in the areas of gene expression analysis and genotyping for point mutations, single nucleotide polymorphisms (SNPs), and short tandem repeats (STRs). Additional methods include interference RNA microarrays and combinations of microarrays and other methods such as laser capture micro-dissection (LCM), comparative genomic hybridization (CGH) and chromatin immunoprecipitation (ChiP). See, e.g., He et al. (2007) Adv. Exp. Med. Biol. 593: 117-133 and Heller (2002) Annu. Rev. Biomed. Eng. 4: 129-153. Other methods include PCR, xMAP, invader assay, mass spectrometry, and pyrosequencing (Wang et al. (2007) Microarray Technology and Cancer Gene Profiling Vol 593 of book series Advances in Experimental Medicine and Biology, pub. Springer New York). Another detection method is allele specific hybridization using probes overlapping the polymorphic site and having about 5, or alternatively 10, or alternatively 20, or alternatively 25, or alternatively 30 nucleotides around the polymorphic region. For example, several probes capable of hybridizing specifically to the genetic variant of interest are attached to a solid phase support, e.g., a “chip”. Oligonucleotide probes can be bound to a solid support by a variety of processes, including lithography. Mutation detection analysis using these chips comprising oligonucleotides, also termed “DNA probe arrays” is described e.g., in Cronin et al. (1996) Human Mutation 7′:244.

In other detection methods, it is necessary to first amplify at least a portion of the gene prior to identifying the allelic variant. Amplification can be performed, e.g., by PCR and/or LCR or other methods well known in the art.

In some cases, the presence of the specific allele in DNA from a subject can be shown by restriction enzyme analysis. For example, the specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230: 1242). In general, the technique of “mismatch cleavage” starts by providing duplexes formed by hybridizing a probe, e.g., RNA or DNA, which is optionally labeled, comprising a nucleotide sequence of the genetic variant of the gene with a sample nucleic acid, obtained from a tissue sample. The double-stranded duplexes are treated with an agent that cleaves single-stranded regions of the duplex such as duplexes formed based on base pair mismatches between the control and sample strands. For instance, RNA/DNA duplexes can be treated with RNase and DNA/DNA hybrids treated with SI nuclease to enzymatically digest the mismatched regions. Alternatively, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249; Cotton et al. (1988) Proc. Natl. Acad. Sci. USA 85:4397; Saleeba et al. (1992) Meth.Enzymol. 217:286-295.

Alterations in electrophoretic mobility may also be used to identify the particular allelic variant. For example, single strand conformation polymorphism (SSCP) may be used to detect differences in electrophoretic mobility between mutant and wild type nucleic acids (Orita et al. (1989) Proc Natl. Acad. Sci USA 86:2766; Cotton (1993) Mutat. Res. 285: 125-144 and Hayashi (1992) Genet. Anal. Tech. Appl. 9:73-79). Single-stranded DNA fragments of sample and control nucleic acids are denatured and allowed to re-nature. The secondary structure of single-stranded nucleic acids varies according to sequence; the resulting alteration in electrophoretic mobility enables the detection of even a single base change. The DNA fragments may be labeled or detected with labeled probes. The sensitivity of the assay may be enhanced by using R A (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

The identity of the genetic variant may also be obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 by of high-melting GC-rich DNA by PCR. In a further embodiment, a temperature gradient is used in place of a denaturing agent gradient to identify differences in the mobility of control and sample DNA (Rosenbaum and Reissner (1987) Biophys. Chem. 265: 1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324: 163); Saiki et al. (1989) Proc. Natl. Acad. Sci. USA 86:6230). Such allele specific oligonucleotide hybridization techniques are used for the detection of the nucleotide changes in the polymorphic region of the gene. For example, oligonucleotide probes having the nucleotide sequence of the specific genetic variant are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotide reagents used as primers for specific amplification may carry the genetic variant of interest in the center of the molecule (so that amplification depends on differential hybridization) (Gibbs et al. (1989) Nucl. Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for PRobeOligo Base Extension. In addition it may be desirable to introduce a novel restriction site in the region of the mutation to create cleavage-based detection (Gasparini et al. (1992) Mol. Cell. Probes 6: 1).

In another embodiment, identification of the genetic variant is carried out using an oligonucleotide ligation assay (OLA), as described, e.g., in U.S. Pat. No. 4,998,617 and in Laridegren, U. et al. Science 241: 1077-1080 (1988). The OLA protocol uses two oligonucleotide probes which are designed to specifically hybridize to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. USA 87:8923-8927). In this method, PCR is used to achieve the exponential amplification of target DNA, which is then detected using OLA. In a variation of the OLA method, as described in Tobe et al. (1996) Nucleic Acids Res. 24: 3728, each allele specific primer is labeled with a unique hapten, i.e. digoxigein, florescein Alexa Fluor 405 SE, Alexa Fluor 488 SE, Alexa Fluor 488 SE, Alexa Fluor 488, 5-TFP, Alexa Fluor 488, 5-SDP, 3-Amino-3-deoxydigoxigenin hemisuccinamide SE, Biotin-X SE, Biotin-XX SE, Biotin-X SSE, Biotin-XX SSE, BODIPY FL-X SE, BODIPY FL STP ester, Cascade Blue acetyl azide, Dansyl-X, SE, DNP-X SE, DNP-X-biocytin-X SE, Fluorescein 5(6)-SFX, Fluorescein-EX SE, Lucifer yellow iodoacetamide, Oregon Green 488-X SE, 5(6)-TAMRA-X SE, Rhodamine Red-X SE, Texas Red-X SE, and each OLA reaction is detected using hapten specific antibodies labeled with reporter enzymes.

The invention provides methods for detecting the genetic variants in Table 2. Because single nucleotide polymorphisms are flanked by regions of invariant sequence, their analysis requires no more than the determination of the identity of the single variant nucleotide and it is unnecessary to determine a complete gene sequence for each patient. Several methods have been developed to facilitate the analysis of SNPs.

The single base polymorphism can be detected by using a specialized exonuclease-resistant nucleotide, as disclosed, e.g., in U.S. Pat. No. 4,656,127. According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

A solution-based method may also be used to determine the identity of the nucleotide of the polymorphic site (as described for example in PCT Patent application publication WO 91/02087). As above, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer. An alternative method is described in PCT Patent application publication WO 92/15712. This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. The method is usually a heterogeneous phase assay, in which the primer or the target molecule is immobilized to a solid phase.

Many other primer-guided nucleotide incorporation procedures for assaying polymorphic sites in DNA have been described (Komher, J. S. et al. (1989) Nucl. Acids. Res. 17:7779-7784; Sokolov, B. P. (1990) Nucl. Acids Res. 18:3671; Syvanen, A.-C, et al. (1990) Genomics 8:684-692; Kuppuswamy, M. N. et al. (1991) Proc. Natl. Acad. Sci. USA 88: 1143-1147; Prezant, T. R. et al. (1992) Hum. Mutat. 1: 159-164; Ugozzoli, L. et al. (1992) GATA 9: 107-112; Nyren, P. et al. (1993) Anal.Biochem. 208: 171-175). These methods all rely on the incorporation of labeled deoxynucleotides to discriminate between bases at a polymorphic site.

Moreover, it will be understood that any of the above methods for detecting alterations in a gene or gene product or polymorphic variants can be used to monitor the course of treatment or therapy.

The methods described herein may be performed, for example, by utilizing pre-packaged diagnostic kits, such as those described below, comprising oligonucleotide reagents which may for genotyping a subject, e.g., analyzing one or more of the genetic variants listed in Table 2, to determine a genetically determined ULN CK level for the subject.

Oligonucleotide Reagents

Probes or primers can be used in the manufacture of microarrays (arrays) for the detection and/or amplification of specific nucleic acids. Primers or probes can be conjugated to a solid surface either in a planar or spherical, among other possibilities, form in the manufacture of devices for simultaneously genotyping a plurality of genetic variants. A variety of such devices are well known in the art.

Oligonucleotides may be synthesized by the sequential addition (5′-3′ or 3′-5′) of activated monomers to a growing chain, which may be linked to an insoluble support. Numerous methods are known in the art for synthesizing oligonucleotides for subsequent individual use or as a part of the insoluble support, for example in arrays (BERNFIELD M R. and ROTTMAN F M. J. Biol. Chem. (1967) 242(18):4134-43; SULSTON J. et al. PNAS (1968) 60(2):409-415; GILLAM S. et al. Nucleic Acid Res. (1975) 2(5):613-624; BONORA G M. et al. Nucleic Acid Res. (1990) 18(11):3155-9; LASHKARI DA. et al. PNAS (1995) 92(17):7912-5; MCGALL G. et al. PNAS (1996) 93(24): 13555-60; ALBERT T J. et al. Nucleic Acid Res. (2003) 31(7):e35; GAO X. et al. Biopolymers (2004) 73(5):579-96; and MOORCROFT M J. et al. Nucleic Acid Res. (2005) 33(8):e75). In general, oligonucleotides are synthesized through the stepwise addition of activated and protected monomers under a variety of conditions depending on the method being used. Subsequently, specific protecting groups may be removed to allow for further elongation and subsequently and once synthesis is complete all the protecting groups may be removed and the oligonucleotides removed from their solid supports for purification of the complete chains if so desired.

A variety of tools can be used to design or select oligonucleotide reagents for a genotyping assay. Tools known in the art, such as Primer3 (http://bioinfo.ut.ee/primer3-0.4.0/), and PrimerQuest (http://www.idtdna.com/Primerquest/Home/Index). These tools can be used to select primers or probes, oligonucleotide reagents, based on optimization of 3 features: melting temperature (Tm), percentage of guanine/cytosine and nucleotide length. Preferably oligonucleotide reagents for use in the present invention have a Tm in the range of 52-58° C.

Preferably oligonucleotide reagents for use in the present invention are 18-30 bases in length.

A 17-mer or longer oligonucleotide reagent should be complex enough so that the likelihood of annealing to sequences other than the chosen target is very low. Oligonucleotide reagents of this length generally are unique sequences in the human genome. It is also important to ensure that portions of the primer do not have sequence or cross-homology with the target. Computer programs such as NCBI Basic Local Alignment Search Tool (BLAST) can be used to find regions of local similarity between sequences. Oligonucleotide reagents longer than 30 bases typically do not demonstrate higher specificity.

Preferably oligonucleotide reagents have a guanine/cytosine content of between 40% and 60% to ensure stable binding to the target nucleotide. The presence of G or C bases at the 3′ end of an oligonucleotide reagent helps to promote correct binding at the 3′ end due to the stronger hydrogen bonding of G and C bases.

A variety of tools are available on line for designing oligonucleotide reagents such as:
https://www.lifetechnologies.com/ca/en/home/products-and-services/product-types/primers-oligos-nucleotides/applied-biosystems-custom-primers-probes/custom-taqman-probes.html and
http://www.bio-rad.com/en-ca/product/per-primers-probes-panels.

Preferably, for the purpose of the present inventions a probe is a polynucleotide of preferably of 15 to 30 nucleotides in length suitable for selective hybridization to an oligonucleotide comprising SEQ. ID. NO. 1, 2, 3, 4, 5, 6, 7, 8, 9, 10, 11, 12, 13, 14, 15, 16, 17, 18, 19 or 20, or a fragment there of comprising a genetic variant of interest, i.e. those listed in Table 2. The length of the probe used will depend, in part, on the nature of the assay used and the hybridization conditions employed.

Oligonucleotide reagents, primers or probes, for use in genotyping the genetic variants listed in Table 2, are synthetic nucleotide sequences that are complimentary to and hybridize to a contiguous sequence within SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, SEQ. ID. NO. 9, SEQ. ID. NO. 10, SEQ. ID. NO. 11, SEQ. ID. NO. 12, SEQ. ID. NO. 13, SEQ. ID. NO. 14, SEQ. ID. NO. 15, SEQ. ID. NO. 16, SEQ. ID. NO. 17, SEQ. ID. NO. 18, SEQ. ID. NO. 19, or SEQ. ID. NO.20. Further, oligonucleotide reagents, for detecting the genetic variants listed in Table 2, have a length of 10 to 50 nucleotides or in other aspects from 12 to 25 nucleotides. Further oligonucleotides, for detecting the genetic variants listed in Table 2, are homologous with a region adjacent to or encompassing a minor allele at SNP site selected from rs1967309, rs12595857, rs2239310, rs11647828, rs8049452, rs12935810, rs74702385, rs17136707, rs8061182, rs111590482, rs4786454, rs2283497, rs2531967, rs3730119 and rs13337675, preferably rs1967309. Further oligonucleotides, for detecting the genetic variants listed in Table 2, are complimentary to a target region of SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, SEQ. ID. NO. 9, SEQ. ID. NO. 10, SEQ. ID. NO. 11, SEQ. ID. NO. 12, SEQ. ID. NO. 13, SEQ. ID. NO. 14, SEQ. ID. NO. 15, SEQ. ID. NO. 16, SEQ. ID. NO. 17, SEQ. ID. NO. 18, SEQ. ID. NO. 19, or SEQ. ID. NO.20 that either comprises a SNP of interest (those indicated in Table 2) or that is adjacent to a SNP of interest (those indicated in Table 2). Probes are at least 85% homologous to the target region, preferably at least 90% identical and more preferably at least 95% identical.

In other aspects a primer comprises 100 or fewer nucleotides, in certain aspects from 12 to 50 nucleotides or from 12 to 30 nucleotides. The primer is at least 70% identical to the contiguous sequence or to the complement of the contiguous nucleotide sequence, preferably at least 80% identical, and more preferably at least 90% identical.

An oligonucleotide reagent for use in the genotyping methods of the invention is between 10-60 nucleotides in length, preferably between 12 and 40 nucleotides in length and more preferably 12 to 25 nucleotides in length. These oligonucleotide reagents are complimentary to region of a oligonucleotide listed in Table 3, SEQ. ID. NOs. 1, 2, 3, 4, 5, 6, 7, 8, 9, 10, 11, 12, 13, 14, 15, 16, 17, 18, 19 and 20 and bind to natural genomic oligonucleotides comprising all or a fragment of these sequences. The degree of complimentary between a oligonucleotide reagent, useful in the genotyping methods of the invention, and SEQ. ID. NO. 1, 2, 3, 4, 5, 6, 7, 10 8, 9, 10 , 11, 12 ,13, 14, 15, 16, 17, 18, 19, or 20 maybe 100%, 95%, 90%, 85% or 80%. For an oligonucleotide to be useful in the genotyping methods of the invention the degree of complimentarity is sufficiently high to allow for specific binding of the oligonucleotide reagent to a region of SEQ. ID. NO. 1, 2, 3, 4, 5, 6, 7, 8, 9, 10, 11, 12, 13, 14, 15, 16, 17, 18, 19 or 20 under high stringency conditions.

Oligonucleotide reagents, including probes and primers, “specific for” a genetic allele bind either to the polymorphic region of a gene or bind adjacent to a polymorphic region of interest. For oligonucleotides that are to be used as primers for amplification, primers are adjacent if they are sufficiently close to be used to produce a polynucleotide comprising the polymorphic region. In one embodiment, oligonucleotides are adjacent if they bind within about 1-2 kb, e.g. less than 1 kb from the polymorphism. Specific oligonucleotides are capable of hybridizing to a sequence, and under suitable conditions will not bind to a sequence differing by a single nucleotide.

Probes are frequently labeled or tagged to enable detection of complexes formed following hybridization with a target nucleotide. Probes can be labeled with radioactive isotopes which are incorporated into the probe during synthesis. Alternately radioactive isotopes can be conjugated to the 5′ or 3′ end of an oligonucleotide post synthesis using enzyme-catalyzed reactions. Commonly used labels or tags used in genotyping include: radioactive isotopes of phosphorus such as ³²P incorporated into the phosphodiester bond of the probe; digoxigenin; biotin; fluorescent dyes and the like. Labeled or tagged probes can then be immobilized on a solid support to manufacture a device for use in a specific genotyping assay. Probes can be labeled by nick translation, Klenow fill-in reaction, PCR or other methods known in the art. Probes of the present invention, their preparation and/or labeling are described in Sambrook et al. (2012) supra.

Oligonucleotide reagents of the invention, whether used as probes or primers, can be detectably labeled. Labels can be detected either directly, for example for fluorescent labels, or indirectly. Indirect detection can include any detection method known to one of skill in the art, including biotin-avidin interactions, antibody binding and the like. Generic modifications of the 5′ end of an oligonucleotide reagent include: biotin, amine, phosphate, aldehyde or thiol groups. Fluorescent dyes are commonly conjugated to the 5′ end of an oligonucleotide reagents include: fluorescein, HEX, ROX, TET, TAMRA. Molecular probe dyes commonly conjugated to to the 5′ end of oligonucleotide reagents include: Alexa Fluor 488*, Alexa Fluor 532*, Alexa Fluor 546*, Alexa Fluor 555*, Alexa Fluor 594*, Alexa Fluor 647*, Alexa Fluor 660*, Alexa Fluor 750*, BODIPY® FL*, BODIPY® 530/550*, BODIPY® 493/503*, BODIPY® 558/569*, 15 BODIPY® 564/570*, BODIPY® 576/589*, BODIPY® 581/591*, BODIPY® FL-X*, BODIPY® TR-X*, BODIPY® TMR*, BODIPY® R6G*, BODIPY® R6G-X*, BODIPY® 630/650*, BODIPY® 650/665*, CASCADE BLUE™ Dye*, MARINA BLUE™ Dye*, OREGON GREEN® 514*, OREGON GREEN® 488*, OREGON GREEN® 488-X*, PACIFIC BLUE™ Dye*, RHODAMINE GREEN™ Dye*, RHODOL GREEN™ Dye*, RHODAMINE GREEN™-X*, RHODAMINE RED™-X*, TEXAS RED®-X*, TAMRA*. The generic modifications, fluorescent dyes or molecular probes provided herein can be conjugated to the 5′ end of probes or primers for use in genotyping methods of the present invention. The oligonucleotides of the invention include oligonucleotides containing modified backbones or non-natural inter-nucleoside linkages. Oligonucleotides having modified backbones include those retaining a phosphorus atom in the backbone, and those that do not have a phosphorus atom in the backbone. Preferred modified oligonucleotide backbones include phosphorothioates or phosphorodithioate, chiral phosphorothioates, phosphotriesters and alkyl phosphotriesters, aminoalkylphosphotriesters, methyl and other alkyl phosphonates including methylphosphonates, 3′-alkylene phosphonates and chiral phosphonates, phosphinates, phosphoroamidates or phosphordiamidates, including 3′-amino phosphoroamidate and aminoalkylphosphoroamidates, and phosphorodiamidatemorpholino oligomers (PMOs), thiophosphoroamidates, phosphoramidothioates, thioalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. Internal modifications can be made within the nucleotide sequence of an oligonucleotide reagent including use of non-natural alternative bases such as deoxyuricil, deoxyinosine, phosphothiates, A-phosphorothioate, G-phosphorothioate, and T-phosphorothioate. Oligonucleotide reagents can also be modified at the 3′ end with biotin or phosphate.

Oligonucleotide reagents can be modified to increase stability by including phosphoramidate, phosphothioate and methylphosphonate analogs within the nucleotide sequence (see also U.S. Pat. Nos. 5,176,996; 5,264,564 and 5,256,775). Primers and probes of the invention can include for example, labeling methylation, inter-nucleotide modification such as pendent moieties (e.g., polypeitides), intercalators (e.g., acridine, psoralen), chelators, alkylators, and modified linkages (e.g., alpha anomeric nucleic acids). Also included are synthetic molecules that mimic nucleotide acid molecules in the ability to bind to a designated sequence by hydrogen bonding and other chemical interactions, including peptide linkages that substitute for phosphate linkages in the nucleotide backbone.

Probes can be used to directly determine the genotype of the sample or can be used simultaneously with or subsequent to amplification. Probes of the present invention, their preparation and/or labeling are described in Sambrook et al. (1989) supra. A probe can be a polynucleotide of any length suitable for selective hybridization to a nucleic acid containing a polymorphic region of the invention. Length of the probe used will depend, in part, on the nature of the assay used and the hybridization conditions employed as described herein.

Labeled probes also can be used in conjunction with amplification of a polymorphism (Holland et al. (1991) Proc. Natl. Acad. Sci. USA 88:7276-7280). U.S. Pat. No. 5,210,015 describes fluorescence-based approaches to provide real time measurements of amplification products during PCR. Such approaches have either employed intercalating dyes (such as ethidium bromide) to indicate the amount of double-stranded DNA present, or they have employed probes containing fluorescence-quencher pairs (also referred to as the “TagMan®” approach) where the probe is cleaved during amplification to release a fluorescent molecule whose concentration is proportional to the amount of double-stranded DNA present. During amplification, the probe is digested by the nuclease activity of a polymerase when hybridized to the target sequence to cause the fluorescent molecule to be separated from the quencher molecule, thereby causing fluorescence from the reporter molecule to appear. The TaqMan® approach uses a probe containing a reporter molecule—quencher molecule pair that specifically anneals to a region of a target polynucleotide i.e. those provided in Table 3 containing a genetic variant of interest i.e. those provided in Table 2.

A plurality of oligonucleotide probes designed for detecting 2 or more of the genetic variants listed in Table 2 can be conjugated to a solid surface. In some embodiments, the surface is silica or glass. In some embodiments, the surface is a metal electrode. Probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence.

A genotyping panel or microarray for use in the genotyping methods of the invention may also comprise primers or probes for detection of genetic variants other than those listed in Table 2 in addition to those listed in Table 2 ,in particular genetic variants known in the art to be associated with the absorption, distribution or metabolism of statins. For example: genetic variants of the COQ2 gene i.e. a minor allele of rs4693596, genetic variants of the SLCO1B1 i.e. a minor allele of rs419056 orrs4363657, genetic variants of the CYPD6 gene i.e. a minor allele of rs35599367.

The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described in U.S. Pat. No. 5,952,172 and by Kelley, S. 0. et al. (1999) Nucl. Acids Res. 27:4830-4837. One or more probes for detecting the genetic variants listed in Table 2 can be affixed to a chip and such a device used to genotype a subject and determine an individualized CK level or ULN CK level and on this basis to diagnose or rule out the presence of statin-induced myopathy in the subject. It is conceivable that probes for detecting the genetic variants listed in Table 2 could be included on a chip with a variety of other probes for uses other than evaluating CK level and diagnosing statin-induced myopathy.

The invention relates to synthetic oligonucleotide molecules, primers and probes that hybridize under high stringency hybridization conditions to naturally occurring oligonucleotides and synthetic oligonucleotides homologous to those in Table 3. Oligonucleotides can be detected and/or isolated by specific hybridization, under high stringency conditions. “High stringency conditions” are known in the art and permit specific hybridization of a first oligonucleotide to a second oligonucleotide where there is a high degree of complimentarity between the first and second oligonucleotide. For the genotyping methods disclosed herein this degree of complimentarity is between 80% and 100% and preferably between 90% and 100%.

The genotype of an individual, the presence or absence of one or more of the genetic variants provided in Table 2, can also be detected from pre-existing data, such as whole genome sequence data present in a data base. The invention provides a computer implemented method of querying genomic data to determine the presence or absence of the genetic variants provided in Table 2.

In particular, the invention also relates to methods and oligonucleotide reagents for determining the presence or absence of the minor alleles listed in Table 2, genotyping individuals using these methods and reagents and determining an ULN CK level for the individual based on the genotype information obtained.

Kits and Devices

As set forth herein, the invention also provides treatment selection methods comprising detecting one or more genetic variants present in Table 2. In some embodiments, the methods use oligonucleotide reagents comprising nucleotide sequences which are complementary to SEQ. ID. NO. 1, 2, 3, 4, 5, 6, 7, 8, 9, 10, 11, 12, 13, 14, 15, 16, 17, 18, 19, or 20. Accordingly, the invention provides kits comprising oligonucleotide reagents for performing the genotyping methods of the invention.

In some embodiments, the invention provides a kit for genotyping an individual, evaluating on-statin CK level in the individual and diagnosing or ruling out the presence of statin-induced myopathy in the individual. Such kits contain one of more oligonucleotide reagents, in particular primers or probes, and instructions for use in the genotyping methods described herein. In one embodiment a kit comprises a plurality of oligonucleotide reagents for genotyping 2 or more of the genetic variants listed in Table 2 by specifically hybridizing to 2 or more of SEQ ID NO 1, 2, 3, 4, 5, 6, 7, 8, 9, 10 ,11, 12, 13, 14, 15, 16, 17, 18, 19, or 20.

Kits for detecting two or more of the genetic variants listed in Table 2, by amplifying at least a portion of two or more fragments of genomic DNA homologues to an oligonucleotide sequence listed in Table 3 i.e. SEQ. ID. NO. 1, 2, 3, 4, 5, 6, 7, 8, 9, 10, 11, 12, 13, 14, 15, 16, 17, 18, 19, and 20, generally comprise two primers per sequence amplified, at least one of which is capable of hybridizing to the genetic variant sequence. Such kits are suitable for detecting a genotype by, for example, fluorescence detection, by electrochemical detection, or by other detection. Yet other kits of the invention comprise at least one reagent necessary to perform the assay. For example, the kit can comprise an enzyme. Alternatively the kit can comprise a buffer or any other necessary reagent.

The kits can include all or some of the positive controls, negative controls, reagents, primers, sequencing markers, probes and antibodies described herein for determining the subject's genotype in the polymorphic region of ADCY9.

In one embodiment the invention provides a genotyping device for genotyping2, 3, 4 or 5 of the genetic variants selected from those listed in Table 2 and comprising multiple oligonucleotide reagents each substantially homologous to an oligonucleotide selected from SEQ. ID. NO. 1, 2, 3, 4, 5, 6, 7, 8, 9, 10, 11, 12, 13, 14, 15, 16, 17, 18, 19, and 20. Genotyping devices of the invention include those described in US 2010/0075296 “Thermal cycling by positioning relative to a fixed-temperature heat source. The invention includes genotyping devices, such as those described in US 2010/0075296, comprising a primer and probe pair for detecting 2 or more of the genetic variants listed in Table 2. In particular, a primer that is designed for polymerase chain reaction amplification and a fluorescently labeled probe, in particular a probe labeled with CalFluor 610, 6-FAM (NHS ester) and 6-FAM (fluorescein).

The following examples are intended merely to illustrate the practice of the present invention and are not provided by way of limitation. Although the present invention has been described herein above by way of preferred embodiments thereof, it can be modified, without departing from the spirit and nature of the subject invention as defined in the appended claim.

### EXAMPLE 1

### Gwas Study

We conducted a genome-wide association study of serum CK levels in 3412 statin-users.
Patients were recruited in Quebec, Canada, and genotyped on Illumina Human610-Quad and an iSelect panel enriched for lipid homeostasis, hypertension and drug metabolism genes. We found a strong association signal between serum levels of CK and the muscle CK (CKM) gene (rs11559024: P=3.69×10⁻¹⁶; R²=0.02) and with the leukocyte immunoglobulin-like receptor subfamily B member 5 (LILRB5) gene (rs2361797: P=1.96×10⁻¹⁰; R²=0.01). Genetic variants in those two genes were independently associated with CK levels in statin users.

Study Population

A discovery cohort was selected based on a secondary phenotype analysis of an existing case-control study for statin-induced myopathy. Demographics of the population are shown in Table 4 GWAS Demographics. The statin case-control study includes 4679 patients recruited in 9 clinical centers throughout the province of Quebec (Canada). The research protocol was approved by the recruiting sites' ethics committees and all human participants gave written informed consent. Cases had documented statin-related muscle symptoms that disappeared upon withdrawal or reduction in dosage or clearly appeared to be statin-related in the opinion of the physician (additional information in Supplementary materials). Controls had dyslipidemia treated with a stable and at least moderate dose of a statin (e.g. atorvastatin ≧20 mg, rosuvastatin ≧20 mg, simvastatin ≧40 mg or pravastatin ≧40 mg) for at least 3 months and had never experienced statin-related side effects. Participants were excluded if they presented with hypothyroidism that is not controlled with a stable dose of supplement for at least the last 3 months; Known hyperthyroidism in the last year; History of alcohol or drug abuse in the last year; Known renal insufficiency with serum creatinine level of 200 μmol/L or more at the time of recruitment; Known severe liver disease with cirrhosis, biliary obstruction, acute or chronic infectious hepatitis at the time of recruitment; Known hereditary or acquired muscle disease; Any medical or psychiatric condition that may make the patient an unsuitable candidate for the study in the physician's opinion; Participation in any other investigational drug study within 30 days of recruitment. Serum CK level was measured at the time of recruitment into the study. The analysis of the case-control study is underway and results will not be presented here. In the present study, we are interested in conducting a secondary phenotype analysis of CK levels with the statin case-control samples. As such, we excluded 954 cases who were not taking a statin at the time of recruitment and 25 subjects because of missing CK measurements or because they had been recruited into the study because of high CK level. There remained 3412 samples for the secondary phenotype analysis of serum CK levels, including 2150 controls (without muscle pain), 814 cases with ongoing pain at the time of recruitment and 448 cases who had experienced muscle pain in the past but did not have pain at the time of recruitment.

Patients with ongoing muscle pain presented with slightly higher mean serum CK levels (129.7±108.4 U/L) than those with a prior history of statin-induced pain (117.1±73.0 U/L) and those tolerating well high-dose statins (115.8±93.7 U/L). In a multiple regression analysis, when adjusting for factors known to influence CK levels including age, sex, BMI and physical activity, we saw a significant difference in CK between cases and controls (P=7.25×10⁻⁷), despite the fact that the majority of CK values were within the normal clinical range. Indeed, only 2 (0.25%) of on-statin patients with ongoing muscle pain had CK values above 3 times the upper limit of normal (ULN) compared to 2 (0.09%) of controls (Table 4).

Genotyping Methods

Whole genome analysis was performed with 620,901 SNPs using the Illumina Infinium HD Assay and the Human 610 quad Bead Chips (Illumina, San Diego, Calif.). The chip was complemented with a custom selection by iSelect design including 11,568 SNPs from candidate genes involved in lipid homeostasis, hypertension and pharmacokinetics. Control samples provided 100% consistency. Following genotype quality procedures and removal of 65 non-Caucasian samples, there remained 584,509 SNPs for analysis and 4391 samples from the case-control statin study.

Statistical Methods

We used the program IMPUTE2 to impute the discovery dataset using reference data sets from the filtered SNPs of the CEU population from including HapMap and 1000 genomes project reference files. Strand alignment was solved by automatically flipping non A/T and C/G SNPs according to position. Ambiguous A/T and C/G SNPs were considered missing and were imputed. We used 568,969 study SNPs, 1,385,414 HapMap SNPs and 7,611,062 SNPs from the 1 000 genomes project. Imputed SNPs with genotype probabilities ≧90% and completion rates of 98% or higher were retained, leaving 3,232,779 SNPs for genome-wide analysis.

Statistical tests performed on genetic data were two-sided and threshold-adjusted for the multiple testing of SNPs. The genome-wide significance threshold P<5×10⁻⁸ was used for the discovery GWAS.¹⁷ CK was log transformed to achieve normality. We used a general linear model (GLM) to test the association of genetic variants with CK levels. Outliers for CK measures were detected using studentized residuals based on the GLM model with selected covariates. In the primary model 3 samples were excluded and 2 for the sensitivity model. In the primary GWAS analysis, adjustment was made for case-control recruitment status to control for sampling bias and adjustment was made for principal components 1 and 2 to control for confounding by population stratification. A sensitivity analysis was also performed with further adjustment for sampling site, physical activity level (mild/moderate/active), age, sex and BMI. The 1-degree of freedom additive genetic test was used for genotypes coded as 0, 1, or 2 according to the number of copies of the minor allele. Reported P values correspond to the likelihood ratio test of the genotype effect, where H₀: β=0; for the additive genetic effect.

Results

Patients taking part of the discovery GWAS with on-statin CK serum levels had a mean age of 62 years, were mostly men (72%) and self-identified as Caucasians (100%). The most frequently used statin was atorvastatin (57%) followed by rosuvastatin (24%). As cases had documented statin-induced muscle symptoms, they were put on lower statin doses and on alternative statin treatment choices, as seen in the lower mean statin dose (P=3.39×10×44) and lower rate of atorvastatin users (41%) in cases as compared to controls (68%).

Patients with ongoing muscle pain presented with slightly higher mean serum CK levels (129.7±108.4 U/L) than those with a prior history of statin-induced pain (117.1±73.0 U/L) and controls tolerating well statins (115.8±93.7 U/L). In a multiple regression analysis, when adjusting for factors influencing CK levels including age, sex, body-mass index and physical activity, we saw a significant difference in CK levels between cases and controls (P=7.25×10−7), despite the fact that the majority of CK values were within the normal clinical range. Indeed, only 2 (0.25%) of on-statin patients with ongoing muscle pain had CK values above 3 times the upper limit of normal (ULN) compared to 2 (0.09%) of controls (Table 4).

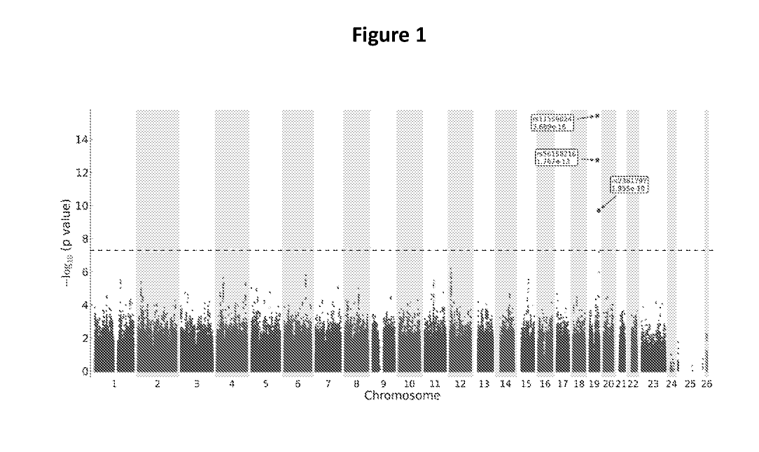

The genome-wide association study (GWAS) for circulating CK levels was conducted as a secondary phenotype analysis of the case-control study of statin-induced myopathy with 3412 statin-users, including 1262 cases with ongoing or past statin-related muscle symptoms and 2150 without. Three SNPs had results passing the significance threshold of 5×10⁻⁸: rs56158216 in the MARK4 gene (P=1.77×10⁻¹³; R²=0.010; MAF=0.01), rs11559024 in the CKM gene (P=3.69×10⁻¹⁶; R₂=0.018; MAF=0.01) and rs2361797 in the LILRB5 gene (P=1.96×10⁻¹⁰; R²=0.010; MAF=0.44) (FIG. 1; Table 5). All three SNPs are located on chromosome 19, at position 45,767,997 (build 37) for rs56158216, which a synonymous SNP in exon 5 of MARK4; at position 45,821,183 for rs11559024 which is a nonsynonymous variant in exon 3 of CKM; and at position 54,753,543 for rs2361797 which is located upstream of gene LILRB5. Rs56158216 in MARK4 is an imputed SNP in linkage disequilibrium with rs11559024 in CKM (r²=0.676, D′=0.847), and rs2361797 in LILRB5 is not in linkage disequilibrium with either of the other two SNPs (r²=0, D′<0.1). LILRB5 SNPs rs406231 (P=1.12×10⁻⁷) was found to be borderline significant (just below the significance threshold of 5×10⁻⁸). The SNP was found to be in linkage disequilibrium with rs2361797 r²=0.64. Homozygotes for the minor allele (CC) at LILRB5 SNP rs406231 had a mean CK of 130.62 (±82.05) U/L compared to 113.33 (±84.83) U/L for homozygotes of the common allele (TT). We conducted a second pass GWAS by further conditioning on the rs11559024 (CKM) and rs2361797 (LILRB5) and found no additional SNPs passing the genome-wide significance threshold and none on chromosome 19 provided P-values less than 10⁻⁵.

When testing for association of the three SNPs with serum CK levels in the cases and controls separately, we can see that the association is strongest in the control group (Table 5.B). SNP rs86158216 in MARK4 had P=4.11×10⁻³ (beta=−0.386) in cases and P=3.53×10⁻⁸ (beta=−0.458) in controls; rs11559024 in CKM had P=3.40×10⁻³ (beta=−0.344) in cases and P=5.18×10⁻¹⁵ (beta=−0.563) in controls; and rs2361797 in LILRB5 had P=2.55×10⁻⁴ (beta=0.021) in cases and P=5.54×10⁻⁷ (beta=0.016) in controls (Table 5.B). We tested for association of the genetic variants with the case-control status alone and in the presence of the modeled covariates and found no association (P<0.05).

Overall, carriers of one copy of the rare allele at rs11559024 in CKM (AG) had a mean (±standard deviation) in CK level of 68.13 (±35.57) U/L, compared to 119.32 (±84.74) U/L for homozygotes for the common allele (AA) (Table 7a). For rs2361797 in LILRB5, homozygotes for the minor allele (TT) had a mean CK level of 129.51 (±92.57) U/L compared to 111.55 (±83.63) U/L for homozygotes of the common allele (CC). Carriers of the G allele at rs11559024 in CKM were more frequent in the lowest tertile of CK values, 67% (48/71), compared to 6% (4/71) in the upper tertile.

In order to investigate the effect of statin dose and statin use duration on the genetic associations with each of the top SNP, those variables were added independently to the multivariate model of CK in the presence of the covariates from the main model. Statin dose or statin use duration added no additional information to this model, were not statistically significant and their interaction effect with the genetic variants were not significant. Non-linear association by generalized additive modeling of rs86158216, rs2361797 and rs11559024 were non-significant.

### EXAMPLE 2

### Biobank Cohort Study 1

Study Population

The findings obtained in the GWAS study were validated in a replication study. Participants for this study were recruited through the Montreal Heart Institute (MHI) Biobank. Statin use and dose was documented at recruitment. For 107 patients, a CK measure taken up to 3 months after recruitment was used. We excluded 322 patients with renal impairment (creatinine levels greater than 200 umol/L). 6009 participants were selected for the analysis, including 3920 with CK measures in statin users and 2089 in non-users. Participants were 98.2% Caucasian and 58.0% male.

For each participant, the most recent CK measure from the hospital records prior to cohort entry was used, excluding CK measurements taken while patients were hospitalized, from emergency visits, from the dialysis clinic, from patients who took part in the discovery cohort and from patients with documented renal impairment (creatinine level greater than 200 μmol/L). 5391 unrelated and Caucasian participants were genotyped.

Genotyping Methods

For the replication of the genetic association with the MHI biobank samples, we used data generated with the Illumina Exome Chip array (version Infinium HumanExome v1.0 DNA Analysis BeadChip). There were 8 variants in the CKM gene and 9 in the LILRB5 gene for which alternative alleles were detected in the dataset. All genotyping was performed at the Beaulieu-Saucier Pharmacogenomics Centre.

Statistical Methods

Serum CK levels were log-transformed to achieve normality. We used a general linear model (GLM) to test the association of genetic markers with CK levels. The 1-degree of freedom additive genetic test was used for genotypes coded as 0, 1, or 2 according to the number of copies of the minor allele. Reported P values correspond to the likelihood ratio test of the genotype effect, where H₀: β=0; for the additive genetic effect.

Results

Replication of the association signal in the CKM and LILRB5 gene was tested by using 8 exome chip variants in the CKM gene including rs11559024 and 9 exomic variants in LILRB5. When testing CK measures in statin users (n=3920), association was detected at variants rs142092440 (P=6.86×10⁻⁴; R²=0.0029) and rs11559024 (P=2.90×10⁻¹⁰; R²=0.010) in the CKM gene, and at variant rs12975366 (P=5.95×10⁻¹¹; R²=0.011) in the LILRB5 gene (Table 6). In sensitivity analyses (n=3238), when adjusting for age, sex, statin dose and physical activity level, the association was maintained with rs142092440 (P=3.43×10⁻⁴), rs11559024 (P=1.39×10⁻⁶) and rs12975366 (P=6.96×10⁻¹⁰). The three genetic variants were not in linkage disequilibrium (r²<0.16). All of the genetic variants were found to contribute independently to serum CK levels.

### EXAMPLE 3

### Biobank Cohort Study 2

Study Population

For the replication cohort, we relied on the Montreal Heart Institute (MHI) Biobank as for Example 2 herein. For each participant, the most recent CK measure from the hospital records prior to cohort entry was used, excluding CK measurements taken while patients were hospitalized, from emergency visits, from the dialysis clinic, from patients who took part in the discovery cohort and from patients with documented renal impairment (creatinine level greater than 200 μmol/L). 5391 unrelated and Caucasian participants were genotyped.

Genotyping Methods

Participants were requited from the Montreal Heart Institute Biobank. 5330 individuals were successfully genotyped for the top 3 discovery SNPs. A single Sequenom panel was designed and validated using HapMap DNA samples. The Sequenom MassArray Maldi-TOF System (Sequenom, La Jolla, Calif.) was used with Sequenom's Typer 4.0.22 Software. Each plate was clustered using autocluster and manually inspected. Two Coriell Institute DNA samples (NA11993 and NA07357) used as controls on each genotyping plate showed 100% concordance on all plate replicates and 100% concordance of genotype calls with expectations for corresponding SNPs in the 1000 Genomes and HapMap reference database.

Statistical Methods

For the replication analysis in the MHI Biobank samples, adjustment was made for age, sex and BMI. The 1-degree of freedom additive genetic test was used for genotypes coded as 0, 1, or 2 according to the number of copies of the minor allele. Reported P values correspond to the likelihood ratio test of the genotype effect, where H0: β=0; for the additive genetic effect.

Results

Replication of the association signal of the statistically significant SNPs in the MARK4, CKM and LILRB5 genes was evaluated by genotyping the SNPs in 5330 participants to the MHI Biobank. In a model that adjusted for age, sex and BMI, all three SNPs were significantly associated with P-values <5×10−8 (Table 9). When restricting the analysis to statin users (n=3389), association was detected with rs56158216 in the MARK4 gene (P=4.22×10−14; R2=0.015), rs11559024 (P=4.32×10−16; R2=0.017) in the CKM gene, and rs2361797 (P=4.45×10−10; R2=0.010) in the LILRB5 gene. The association was similarly strong in statin non-users (n=1941) for all three SNPs with similar effect sizes, as well as in the combined set with rs56158216 (P=1.12×10−18; R2=10 0.013) in the MARK4 gene, rs11559024 (P=1.19×10−21; R2=0.015) in the CKM gene, and rs2361797 (P=1.79×10−17; R2=0.013) in the LILRB5 gene (Table 9). Rs56158216 in MARK4 was in linkage disequilibrium with rs11559024 in CKM (r2=0.728, D′=0.886), and rs2361797 in LILRB5 was not in linkage disequilibrium with either of the other two SNPs (r2<0.01, D′<0.2). In a regression model of CK, when including covariates and conditioning for the rs11559024 CKM variant, the MARK4 SNP rs56158216 lost its association signal (P=0.1874).

The genetic variants in the CKM and LILRB5 gene were found to contribute independently to serum CK levels. In the MHI Biobank, the CKM rs11559024 variant could explain alone 1.7% of the inter-individual variability in CK levels in the 3389 statin-users and 1.2% in 1941 statin non-users; and the LILRB5 rs2361797 could explain 1.0% in of the variability in CK levels in statin-users and 1.7% in statin non-users.

TABLE 4
GWAS Demographics

| | | | | Cases | |
|---|---|---|---|---|---|
| | | All | Controls | With pain | Past pain |
| | | n = 3412 | n = 2150 | n = 814 | n = 448 |
| Age (years) | Mean | 62.40 | 63.24 | 60.67 | 61.50 |
| | (Std) | (±10.32) | (±10.41) | (9.52) | (±10.80) |
| Men | n | 2467 | 1691 | 514 | 262 |
| | (%) | (72.30%) | (78.65%) | (63.14%) | (58.48%) |
| Race - | n | 3412 | 2150 | 814 | 448 |
| Caucasian | (%) | (100.0%) | (100.0%) | (100.0%) | (100.0%) |
| BMI | Mean | 28.83 | 28.85 | 29.04 | 28.41 |
| | (Std) | (±4.95) | (±4.84) | (±4.94) | (±5.47) |
| Creatine | Mean | 119.30 | 115.83 | 129.69 | 117.06 |
| kinase (U/L) | (Std) | (±95.33) | (±93.70) | (108.84) | (±72.98) |
| CK above 3 | n | 4 | 2 | 2 | 0 |
| times ULN | (%) | (0.12%) | (0.09%) | (0.25%) | (0.00%) |
| Physical | n | | | | |
| activity | (%) | | | | |
| Often | | 652 | 402 | 148 | 102 |
| | | (19.11%) | (18.70%) | (18.18%) | (22.77%) |
| Sometimes | | 714 | 461 | 150 | 103 |
| | | (20.93%) | (21.44%) | (18.43%) | (22.99%) |
| Never/rarely | | 2028 | 1278 | 508 | 242 |
| | | (59.44%) | (59.44%) | (62.41%) | (54.02%) |
| Tobacco | | | | | |
| Current | n | 388 | 261 | 83 | 44 |
| smoker | (%) | (11.37%) | (12.14%) | (10.20%) | (9.82%) |
| Past smoker | n | 1951 | 1249 | 464 | 238 |
| | (%) | (57.18%) | (58.09%) | (57.00%) | (53.13%) |
| Diabetes | n | 750 | 540 | 136 | 74 |
| | (%) | (21.98%) | (25.12%) | (16.71%) | (16.52%) |
| Hypertension | n | 2047 | 1350 | 463 | 234 |
| | (%) | (59.99%) | (62.79%) | (56.88%) | (52.23%) |
| Dyslipidemia | n | 3355 | 2112 | 803 | 440 |
| | (%) | (98.33%) | (98.23%) | (98.65%) | (98.21%) |
| Peripheral | n | 331 | 216 | 68 | 47 |
| vascular | (%) | (9.70%) | (10.05%) | (8.35%) | (10.49%) |
| disease | | | | | |
| Previous | n | 1016 | 725 | 187 | 104 |
| myocardial | (%) | (29.78%) | (33.72%) | (22.97%) | (23.21%) |
| infarction | | | | | |
| Stroke/TIA | n | 204 | 123 | 55 | 26 |
| | (%) | (5.98%) | (5.72%) | (6.76%) | (5.80%) |
| Angina | n | 1307 | 885 | 271 | 151 |
| | (%) | (38.31%) | (41.16%) | (33.29%) | (33.71%) |
| Previous | n | 1003 | 704 | 189 | 110 |
| PCI | (%) | (29.40%) | (32.74%) | (23.22%) | (24.55%) |
| Previous | n | 678 | 489 | 129 | 60 |
| CABG | (%) | (19.87%) | (22.74%) | (15.85%) | (13.39%) |
| Congestive | n | 112 | 87 | 15 | 10 |
| Heart failure | (%) | (3.28%) | (4.05%) | (1.84%) | (2.23%) |
| Current statin | | | | | |
| Atorvastatin | n | 1972 | 1453 | 367 | 152 |
| | (%) | (57.80%) | (67.58%) | (45.09%) | (33.93%) |
| Rosuvastatin | n | 823 | 400 | 275 | 148 |
| | (%) | (24.12%) | (18.60%) | (33.78%) | (33.04%) |
| Simvastatin | n | 328 | 194 | 87 | 47 |
| | (%) | (9.61%) | (9.02%) | (10.69%) | (10.49%) |
| Pravastatin | n | 215 | 91 | 54 | 70 |
| | (%) | (6.30%) | (4.23%) | (6.63%) | (15.63%) |
| Fluvastatin | n | 58 | 5 | 25 | 28 |
| | (%) | (1.70%) | (0.23%) | (3.07%) | (6.25%) |
| Lovastatin | n | 16 | 7 | 6 | 3 |
| | (%) | (0.47%) | (0.33%) | (0.74%) | (0.67%) |
| Statin dose | Mean | 44.74 | 49.81 | 37.18 | 34.19 |
| (atorvastatin | (Std) | (±39.05) | (±39.60) | (±36.30) | (±36.91) |
| equivalent, mg) | | | | | |

TABLE 5
GWAS Results (Example 1) SNPs with P < 5 × 10⁻⁸.
A. Discovery GWAS results (P < 5 × 10⁻⁸)

Discovery GWAS

| Chr | Position | SNP | Gene | Alleles | MAF | N | Beta | SE | P value | R² |
|---|---|---|---|---|---|---|---|---|---|---|
| 19 | 45,767,997 | rs56158216* | MARK4 | T/C | 0.008 | 3326 | 0.468 | 0.063 | 1.77 × 10⁻¹³ | 0.0097 |
| 19 | 45,767,997 | rs56158216† | MARK4 | T/C | 0.008 | 3326 | −0.441 | 0.071 | 4.53 × 10⁻¹⁰ | 0.0097 |
| 19 | 45,821,183 | rs11559024 | CKM | G/A | 0.010 | 3384 | −0.501 | 0.061 | 3.69 × 10⁻¹⁶ | 0.0179 |
| 19 | 54,753,543 | rs2361797 | LILRB5 | T/C | 0.443 | 3386 | 0.080 | 0.013 | 1.96 × 10⁻¹⁰ | 0.0101 |

B. By subgroup of the discovery population for SNPs in panel A

| | Cases (past | | | | | | | | | | | |
|---|---|---|---|---|---|---|---|---|---|---|---|---|
| | and current pain) | | | Cases (past pain) | | | Cases (current pain) | | | Controls | | |
| SNP | n | Beta (SE) | P value | n | Beta (SE) | P value | n | Beta (SE) | P value | n | Beta (SE) | P value |
| rs56158216† | 1226 | −0.386 (0.134) | 0.00411 | 436 | −0.371 (0.226) | 0.10121 | 790 | −0.457 (0.169) | 0.00690 | 2100 | −0.458 (0.083) | 3.53 × |
| | | | | | | | | | | | | 10⁻⁸ |
| rs11559024 | 1248 | −0.344 (0.117) | 0.00340 | 447 | −0.504 (0.153) | 0.00109 | 801 | −0.213 (0.179) | 0.23550 | 2136 | −0.563 (0.071) | 5.1810⁻¹⁵ |
| rs2361797 | 1250 | 0.078 (0.021) | 0.00026 | 447 | 0.08 (0.035) | 0.02417 | 803 | 0.075 (0.027) | 0.00587 | 2136 | 0.078 (0.016) | 5.5410⁻⁷ |

For both panels A and B:
*Imputed SNP analysed by dosage in PLINK software making use of minor allele as reference allele;
^{†}Imputed SNP analysed in GLM by fixing the most likely alleles;
Position in NCBI build 37;
Alleles: minor/major alleles;
MAF: Minor allele frequency;
Beta is the regression parameter for allelic effect of the genetic term coded as 0, 1, 2 for the number of minor allele by genotype;
SE is the standard error of Beta;
P value is for the genetic term in a multiple regression equation;
R² is the coefficient of determination for the genetic term alone in a simple linear regression;
Primary GWAS analysis is adjusted for 2 principal components for genetic ancestry, case-control myotoxicity status, age, sex sampling site, physical activity level and body-mass index.

TABLE 6
Results Biobank Cohort Study 1 (Example 2), SNPs with P < 0.05.

| | | | | | | | | CK in statin | | | |
|---|---|---|---|---|---|---|---|---|---|---|---|
| | | | | | | CK in statin users | | non-users (n = | | Combined | |
| | | | Protein | | Minor/Major | (n = 3920) | | 2089) | | (n = 6009) | |
| Gene | Position | RS number | change | MAF | allele | P value | R-squared | P value | R-squared | P value | R-squared |
| CKM | Chr19: 45810010 | rs142092440 | *382Q | 0.001 | G/A | 6.86E−04 | 0.0029 | 0.2074 | 0.0008 | 4.98E−04 | 0.0020 |
| CKM | Chr19: 45815163 | rs17357122 | T166M | 0.008 | A/G | 0.0978 | 0.0007 | 0.0280 | 0.0023 | 0.0121 | 0.0010 |
| CKM | Chr19: 45821183 | rs11559024 | E83G | 0.011 | G/A | 2.90E−10 | 0.0101 | 2.88E−09 | 0.0168 | 1.09E−18 | 0.0129 |
| LILRB5 | Chr19: 54759361 | rs12975366 | D247G | 0.436 | G/A | 5.95E−11 | 0.0109 | 3.97E−05 | 0.0081 | 1.11E−14 | 0.0099 |

Position is in build 37;
RS number is from NCBI dbSNP 137;
Protein change from NCBI;
MAF: Minor allele frequency estimated in 6009 Biobank samples;
P value is for the genetic term in a simple linear regression;
R-squared is the coefficient of determination for the genetic term alone in a simple linear regression;
N/A: not available.

TABLE 7
GWAS and Cohort Study 1, Mean Serum CK Values by Genotype
7a. Discovery GWAS (Example 1)

| Gene | Variant | Genotype | N | Mean CK (U/L) | Estimated ULN |
|---|---|---|---|---|---|
| CKM | rs142092440 | GG | 0 | N/A | |
| | | AG | 71 | 68.13 (±35.57) | 126.82 |
| | | AA | 3335 | 119.32 (±84.74) | 259.14 |
| LILRB5 | rs2361797 | TT | 667 | 129.51 (±92.57) | 282.25 |
| | | CT | 1671 | 118.03 (±80.78) | 251.32 |
| | | CC | 1070 | 111.55 (±83.63) | 249.54 |
| LILRB5 | rs406231 | CC | 393 | 130.62 (±82.05) | 266.00 |
| | | AC | 1512 | 120.00 (±84.44) | 259.33 |
| | | AA | 1475 | 113.33 (±84.83) | 253.30 |

7b. Biobank Cohort Study 1 (Example 2)

| | | | Statin users | | Statin non- | | Combined | |
|---|---|---|---|---|---|---|---|---|
| | | | (n = 3920) | | users (n = 2089) | | (n = 6009) | |
| Gene | Variant | Genotype | N | Mean CK (U/L) | N | Mean CK (U/L) | N | Mean CK (U/L) |
| CKM | rs142092440 | GG | 0 | N/A | 0 | N/A | 0 | N/A |
| | | AG | 12 | 48.83 ± 18.45 | 4 | 97.38 ± 91.55 | 16 | 52.13 ± 26.54 |
| | | AA | 3908 | 117.28 ± 127.67 | 2085 | 102.73 ± 110.3 | 5993 | 112.22 ± 122.1 |
| CKM | rs11559024 | GG | 0 | N/A | 0 | N/A | 0 | N/A |
| | | AG | 78 | 77.88 ± 101.33 | 61 | 58.74 ± 47.46 | 139 | 69.48 ± 82.46 |
| | | AA | 3842 | 117.86 ± 127.9 | 2028 | 103.97 ± 111.3 | 5870 | 113.06 ± 122.59 |
| LILRB5 | rs12975366 | GG | 759 | 102.56 ± 102.04 | 416 | 91.91 ± 106.81 | 1175 | 98.79 ± 103.83 |
| | | AG | 1928 | 115.83 ± 131.2 | 1026 | 103.77 ± 115.79 | 2954 | 111.64 ± 126.18 |
| | | AA | 1233 | 127.93 ± 134.75 | 647 | 107.78 ± 102.78 | 1880 | 120.99 ± 125.02 |

7c. Cohort Study 1 - Statin non-users (Example 1)

| | | | Statin non-users | | |
|---|---|---|---|---|---|
| | | | (n = 2089) | | |
| Gene | Variant | Genotype | N | Mean CK (U/L) | Estimated ULN |
| CKM | rs142092440 | GG | 0 | N/A | |
| | | AG | 4 | 97.38 ± 91.55 | 248.44 |
| | | AA | 2085 | 102.73 ± 110.3 | 284.73 |
| CKM | rs11559024 | GG | 0 | N/A | |
| | | AG | 61 | 58.74 ± 47.46 | 137.05 |
| | | AA | 2028 | 103.97 ± 111.3 | 287.62 |
| LILRB5 | rs12975366 | GG | 416 | 91.91 ± 106.81 | 268.15 |
| | | AG | 1026 | 103.77 ± 115.79 | 294.82 |
| | | AA | 647 | 107.78 ± 102.78 | 277.37 |

7d. Cohort Study - combined (Example 1)

| | | | Combined (n = 6009) | | |
|---|---|---|---|---|---|
| Gene | Variant | Genotype | N | Mean CK (U/L) | Estimated ULN |
| CKM | rs142092440 | GG | 0 | N/A | |
| | | AG | 16 | 52.13 ± 26.54 | 95.66 |
| | | AA | 5993 | 112.22 ± 122.1 | 312.46 |
| CKM | rs11559024 | GG | 0 | N/A | |
| | | AG | 139 | 69.48 ± 82.46 | 204.71 |
| | | AA | 5870 | 113.06 ± 122.59 | 314.11 |
| LILRB5 | rs12975366 | GG | 1175 | 98.79 ± 103.83 | 269.07 |
| | | AG | 2954 | 111.64 ± 126.18 | 318.58 |
| | | AA | 1880 | 120.99 ± 125.02 | 326.02 |

Mean ± standard deviation;
N/A: not available.

TABLE 8
Biobank Cohort Study 2 (Example 3): Mean Serum CK Level by Genotype

| | | | Replication cohort | | Replication cohort | | Replication cohort | |
|---|---|---|---|---|---|---|---|---|
| | | | All | | Statin users only | | Statin non-users | |
| Gene | Variant | Genotype | N | Mean CK (U/L) | N | Mean CK (U/L) | N | Mean CK (U/L) |
| MARK4 | rs56158216 | TT | 0 | N/A | 0 | N/A | 0 | N/A |
| | | TC | 125 | 75.32 (±98.97) | 77 | 76.40 (±116.73) | 48 | 73.58 (±61.68) |
| | | CC | 5204 | 106.36 (±89.23) | 3311 | 106.88 (±80.09) | 1893 | 105.44 (±103.32) |
| CKM | rs11559024 | GG | 0 | N/A | 0 | N/A | 0 | N/A |
| | | AG | 116 | 63.28 (±40.42) | 70 | 61.04 (±37) | 46 | 66.7 (±45.33) |
| | | AA | 5212 | 106.6 (±90.16) | 3318 | 107.18 (±81.62) | 1894 | 105.6 (±103.45) |
| LILRB5 | rs2361797 | TT | 905 | 119.82 (±110.65) | 567 | 123.97 (±117.17) | 338 | 112.84 (±98.51) |
| | | CT | 2656 | 106.5 (±81.37) | 1713 | 105.23 (±70.85) | 943 | 108.83 (±97.63) |
| | | CC | 1767 | 97.07 (±88.38) | 1108 | 98.69 (±71.95) | 659 | 94.36 (±110.65) |

Mean (± standard deviation);
N/A: not available.

TABLE 9
Biobank Cohort Study 2 (Example 3)Association between Genetic Variants and Serum CK Levels

| | | | CK in statin users | | | CK in statin | | | | | |
|---|---|---|---|---|---|---|---|---|---|---|---|
| | | | (n = 3389) | | | non-users (n = 1941) | | | Combined (n = 5330) | | |
| SNP | Gene | MAF | Beta (SE) | P value | R² | Beta (SE) | P value | R² | Beta (SE) | P value | R² |
| rs56158216 | MARK4 | 0.012 | −0.486 | 4.22 × 10⁻¹⁴ | 0.0146 | −0.387 (0.083) | 3.18 × 10⁻⁶ | 0.0100 | −0.450 (0.051) | 1.12 × 10⁻¹⁸ | 0.0128 |
| | | | (0.064) | | | | | | | | |
| rs11559024 | CKM | 0.010 | −0.547 | 4.32 × 10⁻¹⁶ | 0.0172 | −0.430 (0.085) | 4.08 × 10⁻⁷ | 0.0117 | −0.505 (0.053) | 1.19 × 10⁻²¹ | 0.0150 |
| | | | (0.067) | | | | | | | | |
| rs2361797 | LILRB5 | 0.419 | 0.088 | 4.45 × 10⁻¹⁰ | 0.0104 | 0.110 (0.018) | 3.17 × 10⁻⁹ | 0.0167 | 0.095 (0.011) | 1.79 × 10⁻¹⁷ | 0.0126 |
| | | | (0.014) | | | | | | | | |

MAF: Minor allele frequency estimated in Biobank samples;
P value is for the genetic term in a multiple regression with adjustment for age, sex, and body mass index;
R²is the coefficient of determination for the genetic term alone in a simple linear regression.

### CONCLUSIONS

We have demonstrated that serum CK measured in statin users is regulated by the genetic background of patients. We found that genetic polymorphisms in the CKM and LILRB5 gene influence circulating CK levels of patients independently of statin-related muscle symptoms. Statin-users who were carriers of the alternative alleles at the CKM SNPs rs142092440 and rs11559024 have significantly lower CK than homozygous carriers of the common allele (P=6.9×10⁻⁴, R²=0.0029; P=2.9×10⁻¹⁰, R²=0.010 respectively). This effect was also observed in non-statin users, as seen in the MHI Biobank samples and where the variance in the estimate of the mean was less than in statin-users.

The genetic variants associated with CK level were found to explain some of the inter-individual variability in CK levels in statin users from the MHI Biobank cohort study. Indeed, genotypes at the CKM rs142092440 variant could explain alone 0.3% of the inter-individual variability in CK; genotypes at the CKM rs11559024 could explain 1.0%; genotypes at the LILRB5 rs12975366 variant could explain 1.1%; and genotypes at the LILRB5 rs2361797 could explain 1.0% of the inter-individual variability in CK.

The examples provided show that the serum CK level in subjects on-statin is associated with specific genetic variants of CKM and LILRB5 genes. Example 1 shows that variant rs11559024 in the CKM gene (Glu83Gly) was significantly associated with CK levels of statin-users (P=3.69×10−16; R2=0.018): carriers of the minor allele (AG) had mean CK levels of 68.1±35.6 U/L while homozygote carriers of the major allele (AA) had values of 119.3±84.7 U/L. The rare allele of rs11559024 was also associated with on-statin CK levels in Biobank Cohort Study 2 (P=4.32×10−16; R2=0.02) and will off-statin CK levels (P=4.08×10−7; R2=0.02).

The GWAS study found that statin-users have significantly different levels of CK between genotype groups of SNP rs2361797 (P=1.96×10−10), a SNP located upstream of the LILRB5 gene. Homozygous carriers (TT) of the minor allele at rs2361797 had higher levels of CK (129.5±92.6 U/L) compared to homozygous carriers of the major allele (CC) (111.6±83.6 U/L). The SNP was not in linkage disequilibrium with the CKM gene variant (r2<0.01). The minor allele had a frequency of 44%, and heterozygote carriers had a mean level of CK that was intermediate between those of the homozygotes. The association was replicated in the MHI Biobank, in statin users (P=4.45×10−10; R2=0.010) and non-users (P=3.17×10−9; R2=0.017). LILRB5 is a member of the leukocyte immunoglobulin-like receptor (LIR) family, which is found in a gene cluster at chromosomal region 19q13.4.25 LIR subfamily B receptors are expressed on immune cells where they bind to MHC class I molecules on antigen-presenting cells and inhibit stimulation of an immune response. The protein is an integral membrane protein with receptor activity and contains four extracellular immunoglobulin-like domains. The protein also includes two immune-receptor tyrosine-based inhibition motifs and three phosphorylation sites in its cytoplasmic part (Hornbeck PV et al, 2004). The LILRB5 gene presents multiple transcript variants encoding different isoforms and is highly expressed in skeletal muscle, liver and gallbladder.²⁷ Mass spectrometry has detected the protein in plasma, liver and aorta and this plasmatic protein has been ascertained in the HUPO plasma proteome project (Uhlen Metal, 2010; Wang M et al, 2012). Currently there is no evidence of LILRB5 modulation by statins. There is no report in the prior art for a possible implication of this gene in human disease, but the implication of immunity in inter-individual variation in CK is not entirely new. Inter-individual variability in CK levels is influenced by the rate of CK leakage from injured muscle fibers into the circulation but it is possible that a portion of the variability is also due to the rate of CK clearance from the circulation (Warren G L et al, Muscle Nerve. 2006; 34:335-346; Ebbeling C B and Clarkson P M, Eur J Appl Physiol. 1990; 60:26-31; Hyatt J P and Clarkson P M, Med Sci Sports Exerc. 1998; 30:1059-1065). CK clearance occurs via the mononuclear phagocytic system in the liver and via Fc receptors that mediate the endocytosis of immune complexes. CK immune complexes are found in the blood and are commonly referred to as macro CK type 1, which is a complex formed by an immunoglobulin, often IgG, and a CK isoenzyme, often CK-BB. 30, 33 LILRB5 variant rs2361797 is an eQTL that has been shown to also have trans effects to the neighbouring genes LILRB3, LILRA6, LILRB2 and TSEN34. 34

The GWAS was in part limited by its reliance on a secondary phenotype analysis approach to the identification of genetic determinants of circulating CK levels. Because cases and controls are selected at different rates, the sample does not constitute a random sample of the general population. As a result, the association tests between the SNPs and CK levels as secondary trait could be distorted in the case-control sample. To our benefit, the sampling rates were not dramatically different, as cases are not rare, and represent 10% of all statin-users. We have excluded 954 participants to the discovery cohort who were past-sufferers of statin-induced myotoxicity and were no longer on statin medication. This could have depleted the sample from the most extreme on-statin CK measures. The MHI Biobank is, however, a representative sample of prevalent statin-users, and the strong replication of the genetic association signals in this cohort adds support for the findings. Although CK was measured on all participants of the discovery cohort at study entry, we had to rely on convenience measures of CK available from hospital records of participants to the MHI Biobank for the replication study. This could add variability in the measure of CK as some measures may have been obtained during acute illness. The discovery study was well powered (80%) to detect genetic determinants of CK levels with effect sizes of R2≧0.014 for a genetic variant with 1% allele frequency, and R2≧0.011 for 44% allele frequency. The GWAS was limited to a low density chip of 600,000 SNPs but with enrichment for selected genetic variants which proved to be a valuable addition as no other CKM gene variants were detected.

1. A method of evaluating blood creatine kinase (CK) levels in a subject having a genome, the method comprising: (i) analyzing the presence or absence in the genome of two or more genetic variants selected from: guanine or cytosine at rs142092440, cytosine or guanine at rs11559024, cytosine or guanine at rs12975366, guanine or cytosine at rs406231 and adenine or thymine at rs2361797, and (ii) determining an upper limit of normal (ULN) CK level for the subject based at least in part on the presence or absence of the genetic variants analyzed.

2. The method of claim 1 wherein the presence or absence of two or more genetic variants is analyzed by querying preexisting genetic sequence data acquired from a biological sample obtained from the subject.

3. The method of claim 1 wherein prior to (i) a biological sample is obtained from the subject and the sample is analyzed for the presence of two or more genetic variants in step (i).

4. (canceled)

5. The method of claim 1 wherein guanine or cytosine at rs142092440 is found to be present and the ULN CK level determined is between 100 and 270 U/L.

6. The method of claim 1 wherein cytosine or guanine at rs11559024 is found to be present and the ULN CK level determined is between 100 and 270 U/L.

7. The method of claim 1 wherein cytosine or guanine at rs12975366 is found to be present and the ULN CK level determined is between 100 U/L and 270 U/L.

8. The method of claim 1 wherein adenine or thymine at rs2361797 is found to be present and the ULN CK level determined is between 300 and 400 U/L.

9. The method of claim 1 wherein guanine or cytosine at rs406231 is found to be present and the ULN CK level determined is between 300 and 400 U/L.

10. The method of claim 1, further comprising obtaining a measure of the subject's in vivo CK level, comparing the in vivo CK level to the ULN CK level determined at (ii) and diagnosing the subject with statin-induced myopathy when the in vivo CK level is greater than the ULN CK level.

11. The method of claim 10 wherein the diagnosis of statin-induced myopathy is determined using the presence or absence of two or more genetic variants as determined in step (I) and one or more factors selected from the group consisting of: sex, age, concomitant drug use and degree of physical activity.

12. The method of claim 3, wherein analyzing comprises nucleic acid amplification.

13. (canceled)

14. The method of claim 3, wherein analyzing is performed using sequencing, 5′ nuclease digestion, molecular beacon assay, oligonucleotide ligation assay, size analysis, single-stranded conformation polymorphism analysis, or denaturing gradient gel electrophoresis (DGGE).

15. The method of claim 3, wherein analyzing is performed using an allele-specific method.

16. The method of claim 15, wherein said allele-specific method is allele-specific probe hybridization, allele-specific primer extension, or allele-specific amplification.

17. The method of claim 3 wherein the biological sample is selected from blood, saliva or buccal cells.

18. The method of claim 10 wherein the in vivo CK level is obtained from pre-existing data or records.

19. The method of claim 10 wherein a biological sample is obtained from the subject prior to assaying the in vivo CK levels, the in vivo CK levels being obtained from the biological sample.

20. The method of claim 1, further comprising assessing a degree of muscular pain in said subject, said subject being diagnosed with statin induced myopathy when the degree of muscular pain is above a predetermined pain threshold.

21. (canceled)

22. (canceled)

23. A kit or device comprising oligonucleotide detection reagents specific for detecting 2 or more minor alleles at a SNP site selected from: rs142092440, rs11559024, rs12975366, rs406231 and rs2361797.

24. The kit or device of claim 23 wherein said oligonucleotide detection reagents are hybridization probes.

25. The kit or device of claim 23 wherein said oligonucleotide detection reagents are primers.

26. (canceled)

27. The kit or device of claim 23 wherein the oligonucleotide detection reagents are conjugated to a solid surface.

28. The kit or device of claim 23 wherein at least one of the oligonucleotide detection reagents is 5′ conjugated to a biotin, aminie, phosphate, aldehyde or thiol group.

29. The kit or device of claim 23 wherein one or more of said oligonucleotide detection reagent is 5′ conjugated to a fluorescent label selected from fluorescein, HEX, ROX, TET, TAMRA,Alexa Fluor 488, Alexa Fluor 532, Alexa Fluor 546, Alexa Fluor 555, Alexa Fluor 594, Alexa Fluor 647, Alexa Fluor 660, Alexa Fluor 750, BODIPY® FL, BODIPY® 530/550, BODIPY® 493/503, BODIPY® 558/569, BODIPY® 564/570, BODIPY® 576/589, BODIPY® 581/591, BODIPY® FL-X, BODIPY® TR-X, BODIPY® TMR, BODIPY® R6G, BODIPY® R6G-X, BODIPY® 630/650, BODIPY® 650/665, CASCADE BLUE™ Dye, MARINA BLUE™ Dye, OREGON GREEN® 514, OREGON GREEN® 488, OREGON GREEN® 488-X, PACIFIC BLUE™ Dye, RHODAMINE GREEN™ Dye, RHODOL GREEN™ Dye, RHODAMINE GREEN™-X, RHODAMINE RED™-X and TEXAS RED®-X.

30. (canceled)

31. (canceled)

32. The kit or device of claim 23 wherein said oligonucleotide detection reagents comprise an alternative base selected from deoxyuricil, deoxyinosine, phosphothiates, A-phosphorothioate, G-phosphorothioate, and T-phosphorothioate.

33. (canceled)

34. (canceled)

35. (canceled)

36. (canceled)

37. (canceled)

38. (canceled)

39. (canceled)

40. (canceled)

41. (canceled)

42. (canceled)

43. The kit or device of claim 23 wherein said detection reagents are selected from: an oligonucleotide of 12 to 30 nucleotides in length and between 90 and 100% homologous with SEQ ID 1, an oligonucleotide of 12 to 30 nucleotides in length and between 90 and 100% homologous with SEQ ID 2, an oligonucleotide of 12 to 30 nucleotides in length and between 90 and 100% homologous with SEQ ID 3, an oligonucleotide of 12 to 30 nucleotides in length and between 90 and 100% homologous with SEQ ID 4, an oligonucleotide of 12 to 30 nucleotides in length and between 90 and 100% homologous with SEQ ID 5, an oligonucleotide of 12 to 30 nucleotides in length and between 90 and 100% homologous with SEQ ID 6, an oligonucleotide of 12 to 30 nucleotides in length and between 90 and 100% homologous with SEQ ID 7, an oligonucleotide of 12 to 30 nucleotides in length and between 90 and 100% homologous with SEQ ID 8, an oligonucleotide of 12 to 30 nucleotides in length and between 90 and 100% homologous with SEQ ID 9, an oligonucleotide of 12 to 30 nucleotides in length and between 90 and 100% homologous with SEQ ID 10, an oligonucleotide of 12 to 30 nucleotides in length and between 90 and 100% homologous with SEQ ID 11, an oligonucleotide of 12 to 30 nucleotides in length and between 90 and 100% homologous with SEQ ID 12, an oligonucleotide of 12 to 30 nucleotides in length and between 90 and 100% homologous with SEQ ID 13, an oligonucleotide of 12 to 30 nucleotides in length and between 90 and 100% homologous with SEQ ID 14, an oligonucleotide of 12 to 30 nucleotides in length and between 90 and 100% homologous with SEQ ID 15, an oligonucleotide of 12 to 30 nucleotides in length and between 90 and 100% homologous with SEQ ID 16, an oligonucleotide of 12 to 30 nucleotides in length and between 90 and 100% homologous with SEQ ID 17, an oligonucleotide of 12 to 30 nucleotides in length and between 90 and 100% homologous with SEQ ID 18, an oligonucleotide of 12 to 30 nucleotides in length and between 90 and 100% homologous with SEQ ID 19, and an oligonucleotide of 12 to 30 nucleotides in length and between 90 and 100% homologous with SEQ ID 20.

44. (canceled)

45. (canceled)

46. (canceled)

47. The method of claim 1 wherein said genetic variants are detected using or more oligonucleotide detection reagents selected from: an oligonucleotide of 12 to 30 nucleotides in length and between 90 and 100% homologous with SEQ ID 1, an oligonucleotide of 12 to 30 nucleotides in length and between 90 and 100% homologous with SEQ ID 2, an oligonucleotide of 12 to 30 nucleotides in length and between 90 and 100% homologous with SEQ ID 3, an oligonucleotide of 12 to 30 nucleotides in length and between 90 and 100% homologous with SEQ ID 4, an oligonucleotide of 12 to 30 nucleotides in length and between 90 and 100% homologous with SEQ ID 5, an oligonucleotide of 12 to 30 nucleotides in length and between 90 and 100% homologous with SEQ ID 6, an oligonucleotide of 12 to 30 nucleotides in length and between 90 and 100% homologous with SEQ ID 7, an oligonucleotide of 12 to 30 nucleotides in length and between 90 and 100% homologous with SEQ ID 8, an oligonucleotide of 12 to 30 nucleotides in length and between 90 and 100% homologous with SEQ ID 9, an oligonucleotide of 12 to 30 nucleotides in length and between 90 and 100% homologous with SEQ ID 10, an oligonucleotide of 12 to 30 nucleotides in length and between 90 and 100% homologous with SEQ ID 11, an oligonucleotide of 12 to 30 nucleotides in length and between 90 and 100% homologous with SEQ ID 12, an oligonucleotide of 12 to 30 nucleotides in length and between 90 and 100% homologous with SEQ ID 13, an oligonucleotide of 12 to 30 nucleotides in length and between 90 and 100% homologous with SEQ ID 14, an oligonucleotide of 12 to 30 nucleotides in length and between 90 and 100% homologous with SEQ ID 15, an oligonucleotide of 12 to 30 nucleotides in length and between 90 and 100% homologous with SEQ ID 16, an oligonucleotide of 12 to 30 nucleotides in length and between 90 and 100% homologous with SEQ ID 17, an oligonucleotide of 12 to 30 nucleotides in length and between 90 and 100% homologous with SEQ ID 18, an oligonucleotide of 12 to 30 nucleotides in length and between 90 and 100% homologous with SEQ ID 19, and an oligonucleotide of 12 to 30 nucleotides in length and between 90 and 100% homologous with SEQ ID 20.
